(19) United States
(12) Patent Application Publication
(10) Pub. No.: US 2009/0318592 A1
(43) Pub. Date: Dec. 24, 2009

(54) Process for the Synthesis of Amine Ethers

(75) Inventors: Markus Frey, Rheinfelden (CH); Valérie Brönnimann, Basel (CH)

Correspondence Address: JoAnn Villamizar;Ciba Corporation/Patent Department, 540 White Plains Road, P.O. Box 2005, Tarrytown, NY, 10591, US

(73) Assignee: CIBA CORPORATION, Tarrytown, NY (US)

(21) Appl. No.: 12/083,052

(22) PCT Filed: Oct. 2, 2006
(86) PCT No.: PCT/EP2006/066962

§ 371 Date: Mar. 4, 2009

(30) Foreign Application Priority Data
Oct. 11, 2005 (EP) 05109432.4

(51) Int. Cl. C08K 5/3492; C07D 211/02; C07D 497/10; C07D 401/02; C07D 401/14; C07F 7/02; C08K 5/3435

(52) U.S. Cl. 524/100; 546/249; 546/19; 544/180; 546/14; 524/99

(57) ABSTRACT

The present invention relates to novel processes for the preparation of a sterically hindered amine ether by reacting a corresponding sterically hindered amine oxide with a ketone or an aldehyde with at least one reactive H in the presence of a peroxydisulphate. Products obtained by this process may be hydrogenated. The compounds made by these processes are particularly effective in the stabilization of polymer compositions against harmful effects of light, oxygen and/or heat and as flame-retardants for polymers.

The present invention relates to a novel process for the preparation of a sterically hindered amine ether by reacting a corresponding sterically hindered amine oxide with a ketone or an aldehyde with at least one reactive H in the presence of a peroxydisulphate. Products obtained by this process may be hydrogenated. The compounds made by these processes are particularly effective in the stabilization of polymer compositions against harmful effects of light, oxygen and/or heat and as flame-retardants for polymers.

WO 01/92228 describes a process for the preparation of amine ethers, e.g. N-hydrocarbyloxy substituted hindered amine compounds, by the reaction of the corresponding N-oxyl intermediate with a hydrocarbon in the presence of an organic hydroperoxide and a copper catalyst.

WO 03/045919 describes a process for the preparation of amine ethers, e.g. N-hydrocarbyloxy substituted hindered amine compounds, by the reaction of the corresponding N-oxyl intermediate with a hydrocarbon in the presence of an organic hydroperoxide and an iodide catalyst.

Reactions of 2,2,6,6-tetramethyl-1-oxopiperidinium chloride with ketones bearing an α-H are described by T. Ren et al. in Bull. Chem. Soc. Jpn., 69, 2935-2941 (1996) and by Y.-C. Liu et al. in Chinese Journal of Chemistry, 14(3), 252-258 (1996).

Ag(I) catalysed oxidation of cyclic ketones by peroxydisulphate ions are described by S. P. Srivastava et al. in Proc. Nat. Acad. Sci. India, 57(A), III, 299-304 (1987).

The preparation of a sterically hindered amine ether by reacting a corresponding sterically hindered amine oxide with a ketone or an aldehyde with at least one reactive H in the presence of a peroxydisulphate may be carried out under mild reaction condition such as a low reaction temperature. For instance, this process is carried out without the use of chlorine or bromine which in the state-of-the-art process are used in stoichiometric amounts to prepare oxo-piperidinium chlorides/bromides from the corresponding amine oxides.

The present invention relates to a process for the preparation of a sterically hindered amine ether which comprises reacting a corresponding sterically hindered amine oxide with a ketone or an aldehyde, preferably a ketone, with at least one reactive H in the presence of a peroxydisulphate. This process may be denoted herein as amine oxide process.

For instance, the ketone or aldehyde contains at least two reactive H.

For example, the reactive H is in α position.

The ketone is for example of the formula

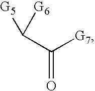

wherein G₇ is as defined below with the exception of H, and G₅ and G₆ are as defined below, preferably the ketone is acetone. The aldehyde is for instance of the formula

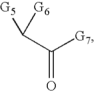

wherein G₇ is H, and G₅ and G₆ are as defined below.

Of interest is a ketone or aldehyde that is symmetrical.

Of interest is an instant process, wherein the obtained product is subsequently hydrogenated. This process may be denoted herein as hydrogenation or hydrogenation process. For instance, the hydrogenation is the reduction of a carbonyl group to a methylene or a hydroxy group.

Of further interest is an amine oxide process, wherein the sterically hindered amine oxide contains at least one group of formula (100)

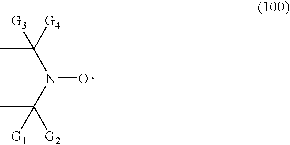

wherein G₁, G₂, G₃ and G₄ are independently alkyl of 1 to 4 carbon atoms or G₁ and G₂ and/or G₃ and G₄ are together tetramethylene or pentamethylene.

Advantageously, the sterically hindered amine oxide used in the amine oxide process is a compound of formula (100a)

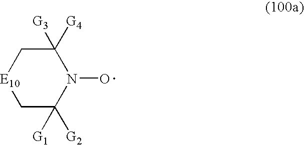

wherein G₁, G₂, G₃ and G₄ are as defined herein;
E₁₀ is a carbon atom which is unsubstituted or substituted by —OH, ═O or by one or two organic residues containing in total 1-500 carbon atoms and optionally 1-200 heteroatoms; the heteroatoms are preferably selected from the group consisting of N, O, S, halogen and Si, in particular of N and O.

An example is an amine oxide or hydrogenation process, wherein the obtained sterically hindered amine ether contains at least one group of formula (101)

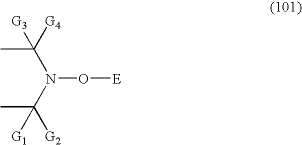

wherein G₁, G₂, G₃ and G₄ are as defined herein;

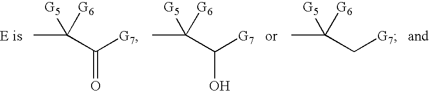

G₅, G₆ and G₇ are independently H, alkyl of 1 to 18 carbon atoms, alkenyl of 2 to 18 carbon atoms, alkinyl of 2 to 18 carbon atoms, aryl of 6 to 10 carbon atoms, aralkyl of 7 to 15 carbon atoms or COO(alkyl) of 2 to 19 carbon atoms; or
G₆ and G₇ form together alkylene of 3 to 10 carbon atoms.

For instance, the obtained sterically hindered amine ether is a compound of formula (101a)

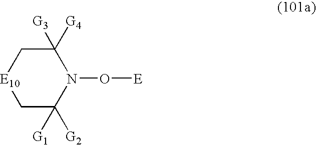

wherein G₁, G₂, G₃ and G₄ and E are as defined herein;
E₁₀ is as defined above with the proviso that E₁₀ can only be a carbon atom substituted by ═O if E is

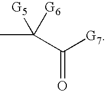

Generally, the product of the amine oxide process is a sterically hindered amine ether containing at least one group of formula (101), wherein E contains a carbonyl group, in particular E is

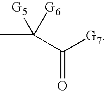

Generally, the product of the hydrogenation process yields a sterically hindered amine ether containing at least one group of formula (101) wherein E contains a hydroxy group or a methylene group, in particular E does not contain a ketone carbonyl group or an aldehyde carbonyl group, especially E does not contain a carbonyl group; for instance E is

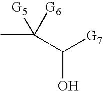

or

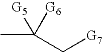

depending on the hydrogenation conditions; an example of E is

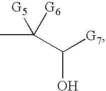

another example of E is

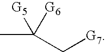

Preferred is an amine oxide or hydrogenation process, wherein G₁, G₂, G₃ and G₄ are methyl.

Of interest is an amine oxide or hydrogenation process, wherein the sterically hindered amine ether is of formula (A) to (O)

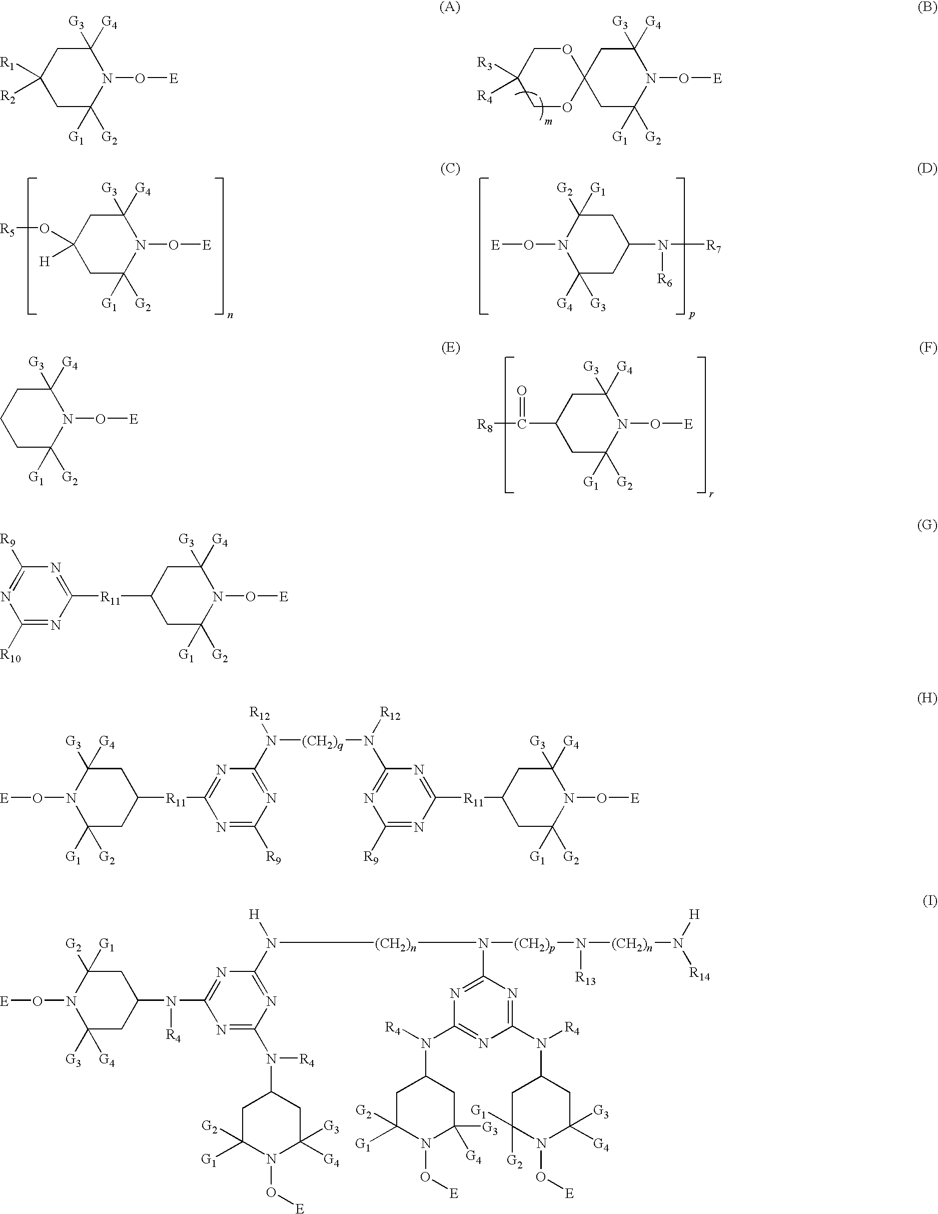

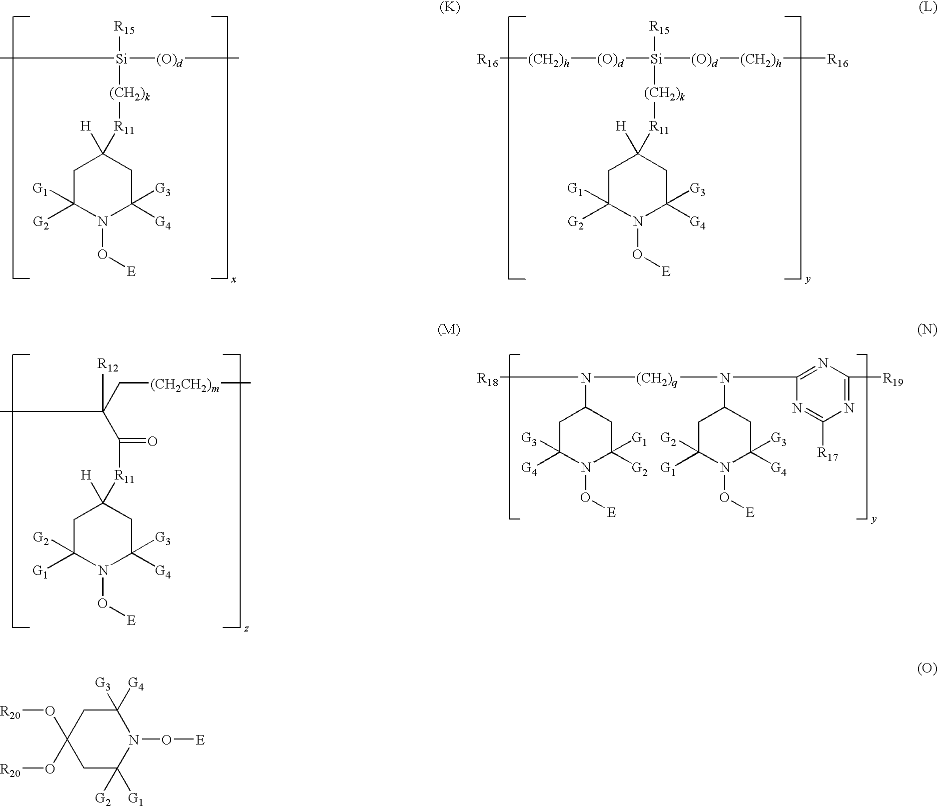

wherein G₁, G₂, G₃ and G₄ are as defined above;
E is as defined herein;
R₁ and R₂ are together ═O when E is

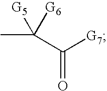

R₁ is H and R₂ is OH when E is

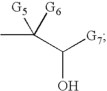

R₁ and R₂ are H when E is

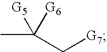

m is 0 or 1;
R₃ is hydrogen, hydroxyl or hydroxymethyl;
R₄ is hydrogen, alkyl of 1 to 12 carbon atoms or alkenyl of 2 to 12 carbon atoms;
n is 1 to 4;
when n is 1,
R₅ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms;
when n is 2,
R₅ is alkylene of 2 to 18 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when n is 3,
R₅ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
when n is 4,
R₅ is a tetravalent acyl radical of an aliphatic or unsaturated aliphatic tetracarboxylic acid, especially 1,2,3,4-butanetetracarboxylic acid, 1,2,3,4-but-2-enetetracarboxylic acid, 1,2,3,5-pentanetetracarboxylic acid and 1,2,4,5-pentanetetracarboxylic acid, or R₅ is a tetravalent acyl radical of an aromatic tetracarboxylic acid containing 10 to 18 carbon atoms;
p is 1 to 3;
R₆ is hydrogen, alkyl of 1 to 18 carbon atoms or acyl of 2 to 6 carbon atoms or phenyl;
when p is 1,
R₇ is hydrogen, phenyl, alkyl of 1 to 18 carbon atoms, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, an acyl radical of an aromatic carboxylic acid containing 7 to 15 carbon atoms, or R₆ and R₇ together are —(CH₂)₅CO—, phthaloyl or a divalent acyl radical of maleic acid;
when p is 2,
R₇ is alkylene of 2 to 12 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when p is 3,
R₇ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
r is 1 to 4;
when r is 1,
R₈ is alkoxy of 1 to 18 carbon atoms, alkenyloxy of 2 to 18 carbon atoms, —NHalkyl of 1 to 18 carbon atoms or —N(alkyl)₂ of 2 to 36 carbon atoms;
when r is 2,
R₈ is alkylenedioxy of 2 to 18 carbon atoms, alkenylenedioxy of 2 to 18 carbon atoms, —NH-alkylene-NH— of 2 to 18 carbon atoms or —N(alkyl)-alkylene-N(alkyl)- of 2 to 18 carbon atoms, or R₈ is 4-methyl-1,3-phenylenediamino;
when r is 3,
R₈ is a trivalent alkoxy radical of a saturated or unsaturated aliphatic triol containing 3 to 18 carbon atoms;
when r is 4,
R₈ is a tetravalent alkoxy radical of a saturated or unsaturated aliphatic tetraol containing 4 to 18 carbon atoms;
R₉ and R₁₀ are independently chlorine, alkoxy of 1 to 18 carbon atoms, —O-T₁, amino substituted by 2-hydroxyethyl, —NH(alkyl) of 1 to 18 carbon atoms, —N(alkyl)T₁ with alkyl of 1 to 18 carbon atoms, or —N(alkyl)₂ of 2 to 36 carbon atoms;
R₁₁ is oxygen, or R₁₁ is nitrogen substituted by either hydrogen, alkyl of 1 to 12 carbon atoms or T₁;

### T₁ is

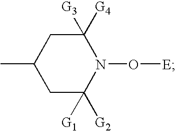

R₁₂ is hydrogen or methyl;
q is 2 to 8;
R₁₃ and R₁₄ are independently hydrogen or the group T₂;

### T₂ is

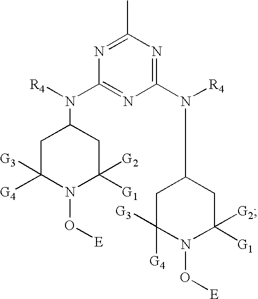

R₁₅ is hydrogen, phenyl, straight or branched alkyl of 1 to 12 carbon atoms, alkoxy of 1 to 12 carbon atoms, straight or branched alkyl of 1 to 4 carbon atoms substituted by phenyl, cycloalkyl of 5 to 8 carbon atoms, cycloalkenyl of 5 to 8 carbon atoms, alkenyl of 2 to 12 carbon atoms, glycidyl, allyloxy, straight or branched hydroxyalkyl of 1 to 4 carbon atoms, or silyl or silyloxy substituted three times independently by hydrogen, by phenyl, by alkyl of 1 to 4 carbon atoms or by alkoxy of 1 to 4 carbon atoms;
R₁₆ is hydrogen or silyl substituted three times independently by hydrogen, by phenyl, by alkyl of 1 to 4 carbon atoms or by alkoxy of 1 to 4 carbon atoms;
d is 0 or 1;
h is 0 to 4;
k is 0 to 5;
x is 3 to 6;
y is 1 to 10;
z is an integer such that the compound has a molecular weight of 1000 to 4000 amu, e.g. z may be from the range 3-10;
R₁₇ is morpholino, piperidino, 1-piperizinyl, alkylamino of 1 to 8 carbon atoms, especially branched alkylamino of 3 to 8 carbon atoms such as tert-octylamino, —N(alkyl)T₁ with alkyl of 1 to 8 carbon atoms, or —N(alkyl)₂ of 2 to 16 carbon atoms;
R₁₈ is hydrogen, acyl of 2 to 4 carbon atoms, carbamoyl substituted by alkyl of 1 to 4 carbon atoms, s-triazinyl substituted once by chlorine and once by R₁₇, or s-triazinyl substituted twice by R₁₇ with the condition that the two R₁₇ substituents may be different;
R₁₉ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃;

### T₃ is

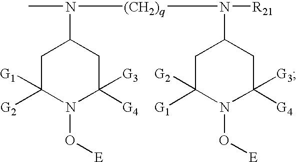

R₂₀ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms; and
R₂₁ is hydrogen, acyl of 2 to 4 carbon atoms, carbamoyl substituted by alkyl of 1 to 4 carbon atoms, s-triazinyl substituted twice by —N(alkyl)₂ of 2 to 16 carbon atoms or s-triazinyl substituted twice by —N(alkyl)T₁ with alkyl of 1 to 8 carbon atoms.

Preferred is an amine oxide or hydrogenation process, wherein the sterically hindered amine ether is of formula (A), (C)-(F), (I) or (N), especially of formula (A), (C), (E) or (N).

More preferred is an amine oxide or hydrogenation process, wherein the sterically hindered amine ether is of formula (A), (C), (E) or (N) and

n is 1 to 2;
when n is 1,
R₅ is hydrogen, alkyl of 1 to 18 carbon atoms, alkenyl of 2 to 18 carbon atoms, glycidyl, 2,3-dihydroxypropyl, an acyl radical of an aliphatic or unsaturated aliphatic carboxylic or carbamic acid containing 2 to 18 carbon atoms;
when n is 2,
R₅ is alkylene of 2 to 18 carbon atoms, a divalent acyl radical of an aliphatic or unsaturated aliphatic dicarboxylic or dicarbamic acid containing 2 to 18 carbon atoms;
q is 4 to 8;
y is 2 to 10;
R₁₇ is alkylamino of 1 to 8 carbon atoms, especially branched alkylamino of 3 to 8 carbon atoms such as tert-octylamino, —N(alkyl)T₁ with alkyl of 1 to 8 carbon atoms, or —N(alkyl)₂ of 2 to 16 carbon atoms;
R₁₈ is hydrogen, s-triazinyl substituted once by chlorine and once by R₁₇, or s-triazinyl substituted twice by R₁₇ with the condition that the two R₁₇ substituents may be different;
R₁₉ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃;

### T₃ is

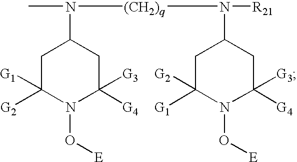

R₂₁ is hydrogen, s-triazinyl substituted twice by —N(alkyl)₂ of 2 to 16 carbon atoms or s-triazinyl substituted twice by —N(alkyl)T₁ with alkyl of 1 to 8 carbon atoms.

Even more preferred is an amine oxide or hydrogenation process, wherein the sterically hindered amine ether is of formula (A), (E) or (N) and

q is 4 to 8;
y is 2 to 10;
R₁₇ is —N(alkyl)T₁ with alkyl of 1 to 8 carbon atoms, or —N(alkyl)₂ of 2 to 16 carbon atoms;
R₁₈ is s-triazinyl substituted twice by R₁₇;
R₁₉ is the group T₃;

### T₃ is

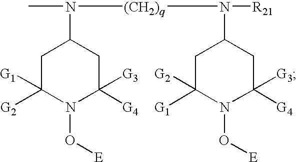

R₂₁ is s-triazinyl substituted twice by —N(alkyl)₂ of 2 to 16 carbon atoms.

Preferably, G₅, G₆ and G₇ are independently H, alkyl of 1 to 18 carbon atoms or alkenyl of 2 to 18 carbon atoms; or

G₆ and G₇ form together alkylene of 3 to 10 carbon atoms.

More preferably, G₅ and G₆ are independently H or as defined for G₇;

G₇ is alkyl of 1 to 8 carbon atoms or alkenyl of 2 to 8 carbon atoms; or
G₆ and G₇ form together alkylene of 3 to 6 carbon atoms.

Most preferably, G₅ and G₆ are independently H or methyl,

G₇ is alkyl of 1 to 3 carbon atoms or alkenyl of 2 to 4 carbon atoms, or
G₆ and G₇ form together alkylene of 4 carbon atoms.

Of interest are G₅ and G₆ that are H. Also of interest is G₇ that is methyl.

Of interest is G₅, G₆ and G₇ that is neither alkenyl nor alkinyl. Also of interest is G₅, G₆ and G₇ that is not COO(alkyl).

For instance, E is

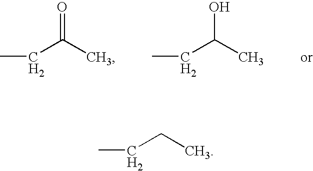

The amine oxide process is generally carried out as follows: A solution of the peroxydisulphate is added at 20-100°, for example 25-80°, in particular 40-70°, especially 50-70° to a mixture of the sterically hindered amine oxide, a ketone or aldehyde, optionally a base and optionally a catalyst A. The product is isolated by saturating the neutralized (pH 7) aqueous phase, separating off the organic layer, removing volatiles and, optionally, purifying the residue thus obtained with conventional techniques such as recrystallization, distillation or chromatography.

The amine oxide process can be run in air (preferred) or in an inert atmosphere such as under nitrogen or argon. The instant process can be run under atmospheric pressure (preferred) as well as under reduced or elevated pressure.

Alternatively, the reactants may be added in another order, in particular the sterically hindered amine oxide may be treated with the peroxydisulphate followed by addition of the ketone or aldehyde.

In the amine oxide process, the reaction temperature may be 20-100°, for example 25-80°, in particular 40-70°, especially 50-70°.

In the amine oxide process, the ketone or aldehyde is usually used in an amount of 1-20 moles, for instance 3-15 moles, especially 6-13 moles, in particular 8-12 moles per mole of nitroxyl moiety.

For instance, the number of nitroxyl moieties corresponds to the number of amine oxide groups; one molecule can contain more than one amine oxide group.

For the amine oxide process, a co-solvents is usually not needed.

For instance, the peroxydisulphate is an alkali metal peroxydisulphate or an ammonium peroxydisulphate. Examples of an alkali metal peroxydisulphate are Na₂S₂O₈ and K₂S₂O₈. Examples of an ammonium peroxydisulphate are (NH₄)₂S₂O₈ and a tetraalkylammonium peroxydisulphate such as (Bu₄N)₂S₂O₈. Preferred is a peroxydisulphate that is Na₂S₂O₈, K₂S₂O₈, (NH₄)₂S₂O₈ or (Bu₄N)₂S₂O₈, especially Na₂S₂O₈.

Typically, the amount of peroxydisulphate is 0.3-3 moles, especially 0.4-1.5 moles, in particular 0.45-1.3 moles, for example 0.5-1.2 moles per mole of nitroxyl moiety.

The peroxydisulphate is usually used dissolved in a solvent, for instance an inorganic solvent such as water or an organic solvent such as a polar protic or a polar non-protic solvent, in particular a solvent with a high dielectric constant (e.g. MeOH, DMF, DMSO, MeCN, sulfolane or acetone). The organic solvent can be used in mixture with water.

The tetraalkylammonium peroxydisulphates are usually used dissolved in an organic solvent. A suitable solvent for Na₂S₂O₈ is water.

The tetraalkylammonium peroxydisulphate may be formed via in situ anion-exchange by a phase-transfer catalyst (e.g. tetraalkylammonium salts) and Na₂S₂O₈, K₂S₂O₈ or (NH₄)₂S₂O₈; this procedure is described by Y. Kim, in Reviews on Heteroatom Chemistry (1999), 20, 69-96.

Examples of tetraalkylammonium salts are tetramethylammonium-, tetraethylammonium-, tributylmethylammonium-, tetrabutylammonium-, tetrahexylammonium- and trioctylmethylammonium-hydroxides, -hydrogensulfates, -fluorides, -acetates, -chlorides, -cyanides, -bromides, -nitrates, -perchlorates and -iodides, preferably tetrabutylammonium hydrogensulphate. The amount of phase-transfer catalyst is for example 0.0001-1.1 moles, especially 0.01-0.2 mole per mole of peroxydisulphate.

The amine oxide process may be carried out in the presence or absence of a catalyst A, preferably, in the presence of a catalyst A.

Catalyst A is typically used in an amount of 0.001-0.5 mole, preferably 0.005-0.1 mole, more preferably 0.01-0.5 mole, most preferably 0.01-0.03 mole per mole of nitroxyl moiety.

For instance, the catalyst A is a salt or a complex of Ag, Mn, Fe, Cu, Co or Ni, especially AgNO₃.

The catalyst A (e.g. AgNO₃) may be added to the reaction mixture as a solid or dissolved in water or a suitable organic solvent such as acetone depending on the solubility of the catalyst A, especially may be added as a solid, in particular may be added dissolved in water.

The cationic charges in the salts and complexes are counterbalanced by anionic ligands commonly known in complex chemistry of transition metals, such as hydride ions (H⁻) or anions derived from inorganic or organic acids, examples being halides, e.g. F⁻, Cl⁻, Br⁻ or I⁻, fluoro complexes of the type BF₄⁻, PF₆⁻, SbF₆⁻ or AsF₆⁻, anions of oxygen acids, alcoholates or acetylides or anions of cyclopentadiene.

Anions of oxygen acids are, for example, sulfate, phosphate, perchlorate, perbromate, periodate, antimonate, arsenate, nitrate, carbonate, the anion of a C₁-C₈carboxylic acid, such as formate, acetate, propionate, butyrate, benzoate, phenylacetate, mono-, di- or trichloro- or -fluoroacetate, sulfonates, for example methylsulfonate, ethylsulfonate, propylsulfonate, butylsulfonate, trifluoromethylsulfonate (triflate), unsubstituted or C₁-C₄alkyl-, C₁-C₄alkoxy- or halo-, especially fluoro-, chloro- or bromo-substituted phenylsulfonate or benzylsulfonate, for example tosylate, mesylate, brosylate, p-methoxy- or p-ethoxyphenylsulfonate, pentafluorophenylsulfonate or 2,4,6-triisopropylsulfonate, phosphonates, for example methylphosphonate, ethylphosphonate, propylphosphonate, butylphosphonate, phenylphosphonate, p-methylphenylphosphonate or benzylphosphonate, carboxylates derived from a C₁-C₈carboxylic acid, for example formate, acetate, propionate, butyrate, benzoate, phenylacetate, mono-, di- or trichloro- or -fluoroacetate, and also C₁-C₁₂-alcoholates, such as straight chain or branched C₁-C₁₂-alcoholates, e.g. methanolate or ethanolate. Also oxides are possible.

Anionic ligands and neutral may also be present up to the preferred coordination number of the complex cation, especially four, five or six. Additional negative charges are counterbalanced by cations, especially monovalent cations such as Na⁺, K⁺, NH₄⁺ or (C₁-C₄ alkyl)₄N⁺.

Suitable neutral ligands are inorganic or organic neutral ligands commonly known in complex chemistry of transition metals. They coordinate to the metal ion through a σ-, π-, μ-, η-type bonding or any combinations thereof up to the preferred coordination number of the complex cation. Suitable inorganic ligands are selected from the group consisting of aquo (H₂O), amino, nitrogen, carbon monoxide and nitrosyl. Suitable organic ligands are selected from the group consisting of phosphines, e.g. (C₆H₅)₃P, (i-C₃H₇)₃P, (C₅H₉)₃P or (C₆H₁₁)₃P, di-, tri-, tetra- and hydroxyamines, such as ethylenediamine, ethylenediaminotetraacetate (EDTA), N,N-dimethyl-N′,N′-bis(2-dimethylaminoethyl)-ethylenediamine (Me₆TREN), N,N′-dimethyl-1,2-benzenediamine, 2-(methylamino)phenol, 3-(methylamino)-2-butanol or N,N′-bis(1,1-dimethylethyl)-1,2-ethanediamine, N,N,N′,N″,N″-pentamethyldiethyltriamine (PMDETA), C₁-C₈-glycols or glycerides, e.g. ethylene or propylene glycol or derivatives thereof, e.g. di-, tri- or tetraglyme, and monodentate or bidentate heterocyclic e⁻ donor ligands.

Heterocyclic e⁻ donor ligands are derived, for example, from unsubstituted or substituted heteroarenes from the group consisting of furan, thiophene, pyrrole, pyridine, bis-pyridine, picolylimine, g-pyran, g-thiopyran, phenanthroline, pyrimidine, bis-pyrimidine, pyrazine, indole, coumarone, thionaphthene, carbazole, dibenzofuran, dibenzothiophene, pyrazole, imidazole, benzimidazole, oxazole, thiazole, bis-thiazole, isoxazole, isothiazole, quinoline, bis-quinoline, isoquinoline, bis-isoquinoline, acridine, chromene, phenazine, phenoxazine, phenothiazine, triazine, thianthrene, purine, bis-imidazole and bis-oxazole.

These ligand complexes are items of commerce or may be formed in situ by mixing a metal salt with the ligand. The amount of ligand may be less than the amount required to completely complex the metal based on its oxidation state. The metal salt or metal-ligand complex may be bound to a solid support such as silica gel so that it can be recovered and reused.

The amine oxide process may be carried out in the presence or absence of a base, for instance in the presence of a base, for example in the absence of a base.

Typically, the amount of base used is 0.1-6 moles, preferably 0.5-4 moles, more preferably 0.5-3 moles, most preferably 0.8-2.5 moles (e.g. of monoprotic base or times 1/n of n-protic base) per mole of peroxydisulphate.

For instance, a monoprotic base is a base that can take up one proton per base molecule. For example, a n-protic base can take up n protons per base molecule.

For example, the base is selected from the group consisting of alkali metal hydroxide, alkaline-earth metal hydroxide, alkali metal hydrogen carbonate, alkaline-earth metal hydrogen carbonate, alkali metal carbonate and alkaline-earth metal carbonate.

For instance, the base is selected from the group consisting of alkali metal hydrogen carbonate and alkali metal carbonate.

Examples of an alkali metal hydroxide are KOH and NaOH. Examples of an alkaline-earth metal hydroxide are Mg(OH)₂ and Ca(OH)₂. Examples of an alkali metal hydrogen carbonate are NaHCO₃ and KHCO₃, especially NaHCO₃. Examples of an alkaline-earth metal hydrogen carbonate are Mg(HCO₃)₂ and Ca(HCO₃)₂. Examples of an alkali metal carbonate are Na₂CO₃ and K₂CO₃, in particular Na₂CO₃. Examples of an alkaline-earth metal carbonate are CaCO₃ and MgCO₃.

The base present in the amine oxide process mixture may be used for in-situ neutralization of the hydrogensulphate formed during the reaction.

An example is an amine oxide process that is carried out in the absence of both a catalyst A and a base. Another example is an amine oxide process that is carried out in the presence of a catalyst A but in the absence of a base. Another example is an amine oxide process that is carried out in the absence of a catalyst A but in the presence of a base. Another example is an amine oxide process that is carried out in the presence of both a catalyst A and a base.

The sterically hindered amine oxides are largely known in the art; they may be prepared by oxidation of the corresponding N—H sterically hindered amine with a suitable oxygen donor, e.g. by the reaction of the corresponding N—H sterically hindered amine with hydrogen peroxide and sodium tungstate as described by E. G. Rozantsev et al., in Synthesis, 1971, 192; or with tert-butyl hydroperoxide and molybdenum (VI) as taught in U.S. Pat. No. 4,691,015, or obtained in analogous manner.

Preferably, the hydrogenation is carried out in the presence of a catalyst B and H₂ or in the presence of a hydrogen equivalent.

Hydrogenation of sterically hindered amine ethers that contain one or more alkenyl and/or alkinyl substituents is preferably carried out in the presence of a hydrogen equivalent, especially NaBH₄.

Hydrogenation of sterically hindered amine ethers that contain one or more alkenyl and/or alkinyl substituents with a catalyst B and H₂ may effect the reduction the alkenyl and/or alkinyl substituents to alkyl substituents.

For example, catalyst B is selected from the group consisting of Pd, Pt, PtO₂, Ru, Rh, Ir, Ni (e.g. Raney, Urushibara) and mixtures thereof and salts thereof and complexes thereof, whereby salts thereof and complexes thereof are less preferred, and the hydrogenation is carried out in the presence or absence of an additive, in particular a Lewis acid such as a halide (e.g. chloride) of B, Al, Si, Sn, Y, In, Ti, Zr, Hf, Sc or La, especially of B, Al, Zr or Ti. The catalyst B is typically loaded on a solid, inert carrier such as carbon, BaSO₄ or CaCO₃. The additive is usually used in an amount of 0.1-5 moles, preferably 0.5-2 moles per mole of sterically hindered amine ether moiety (product of amine oxide process).

For instance, the number of sterically hindered amine ether moieties corresponds to the number of sterically hindered amine ether groups; one molecule can contain more than one sterically hindered amine ether group.

Catalyst B as defined above is usually sufficient for the reduction of a carbonyl group to a hydroxy group and generally an additive is not needed. For the reduction of a carbonyl group to a hydroxy group, the preferred catalyst B is Ru/C.

Catalyst B in presence of an additive is usually needed for the reduction of a carbonyl group to a methylene group. Preferred for this reduction is the catalyst B Pt/C or PtO₂, each in presence of the additive AlCl₃, ZrCl₄ or TiCl₄; especially Pt/C in the presence of TiCl₄.

For instance, the hydrogen equivalent is a borohydride; a borane; an aluminumhydride; a silane; a secondary alkanol in combination with its alkoxide; a metal in combination with a protic solvent; an alkali metal hydride; formic acid, an alkali metal formiate or an ammonium formiate, each in combination with HCOOHₓNEt₃ or Pd/C; phosphinic acid, an alkali metal phosphinate or an ammonium phosphinate, each in combination with NaH₂PO₂ or Pd/C; an alkali metal dithionite; baker's yeast; a borohydride, a borane, an aluminumhydride, a silane or a metal, each in combination with an additive, in particular a Lewis acid such as a halide (e.g. chloride) of B, Al, Si, Sn, Y, In, Ti, Zr, Hf, Sc or La.

The hydrogenation may be the reduction of a carbonyl group to a hydroxy group that can be be carried out as described in R. Larock, Comprehensive Organic Transformations, VCH, New York (1989) and literature cited therein. The hydrogen equivalent is for example as described above in the absence an additive. The preferred hydrogen equivalent for this reduction is NaBH₄.

Examples of a borohydride are alkali and tetraalkylammonium borohydrides (e.g. NaBH₄, KBH₄, LiBH4, Bu₄NBH₄), cyanoborohydrides (e.g. NaBH₃CN, Bu₄NBH₃CN), trialkylborohydrides (e.g. KEt₃BH, K(sec-Bu)₃BH), trialkoxyborohydrides (e.g. Na(MeO)₃BH) and tricarboxyloxyborohydrides (e.g. Na(AcO)₃BH).

Examples of a borane are borane itself and its adducts with N, O and S based Lewis bases (e.g. Et(iPr)₂NxBH₃, THFxBH₃, Me₂SxBH₃) and dialkylboranes (e.g. 9-borabicyclo[3.3.1]nonane).

Examples of an aluminumhydride are alkali aluminumhydrides (e.g. LiAlH₄), trialkoxyaluminumhydrides (e.g. Li(tBuO)₃AlH) and dialkylaluminumhydrides (e.g. (iBu)₂AlH). Examples of a silane are trialkylsilanes (e.g. Et₃SiH), trialkoxysilanes (e.g. (EtO)₃SiH), alkyldialkoxysilanes (e.g. Me(EtO)₂SiH) and oligomers and polymers derived thereof.

An examples of a secondary alkanol in combination with its alkoxide (e.g. of Ti, Al or Zr) are iPrOH and Al(OiPr)₃ (e.g. Meerwein-Ponndorf-Verley conditions).

Examples of a metal (e.g. a strongly reducing metal) in combination with a protic solvent are sodium in EtOH and Zn in aqueous HCl.

An example of an alkali metal hydride is LiH.

An example of an alkali metal dithionite is Na₂S₂O₄.

The hydrogenation may be the reduction of a carbonyl group to a methylene group which can be carried out with the hydrogen equivalent in combination with an additive as described above. The preferred hydrogen equivalent in combination with an additive is for example NaBH₄ in combination with TiCl₄, for instance Et₃SiH in combination with BF₃.

Examples of an aluminumhydride with an additive is LiAlH₄ with InBr₃ (see N. Fu et al., Chinese Chemical Letters (2003), 14(10), 1018-1020; CAN 140:356770) and LiAlH₄ with TiCl₃ (see R. Dams et al., Bulletin des Societes Chimiques Belges (1982), 91(4), 311-319; CAN 97:181268).

Examples of a silane with an additive are Et₃SiH with AlCl₃ (see D. Kursanov et al., Zhurnal Organicheskoi Khimii (1985), 21(11), 2274-2282; CAN 104:185623) and Et₃SiH with BF₃ (see J. Fry et al., Journal of Organic Chemistry (1978), 43(2), 374-375; CAN 88:61663).

An example of a metal (e.g. a strongly reducing metal) with an additive is Zn with AlCl₃ (see P. Chowdhury et al., Journal of Chemical Research, Synopses (1994), 6, 230-231; CAN 121:107588).

For instance, the reduction of a carbonyl group to a hydroxy group with a catalyst B and H₂ in the absence of an additive is carried out as follows:

A mixture of the sterically hindered amine ether (product of amine oxide process) and Ru/C (0.001-0.1 mole, preferably 0.005-0.05 mole, most preferably 0.005-0.02 mole, in particular about 0.02 mole per mole of sterically hindered amine ether moiety) in an organic solvent (e.g. alcohols like methanol, ethanol, in particular methanol) is stirred at 20-100°, preferably at 30-70°, especially at 40-60°; the hydrogen pressure is 1-100 bar, preferably 20-80 bar, more preferably 35-55 bar, especially 40-50 bar. The catalyst is filtered off and the solvent is evaporated. The obtained product is purified by methods known in the art such as distillation.

Typically, the reduction of a carbonyl group to a hydroxy group with a hydrogen equivalent in the absence of an additive is carried out as follows:

A mixture of the sterically hindered amine ether (product of amine oxide process) and NaBH₄ (0.25-2 mole, preferably 0.5-1.7 mole, most preferably 0.9-1.5 mole per mole of sterically hindered amine ether moiety) in an organic solvent (e.g. alcohols like methanol, ethanol; ethers like tetrahydrofuran, diethyl ether, t-butylmethyl ether; esters like ethyl acetate, methyl acetate; dimethylformamide; preferably tetrahydrofuran) is stirred at 0-100° C., preferably 25-80° C. Excess NaBH₄ is destroyed by addition of water. Extraction with ethyl acetate followed by solvent evaporation yields the product.

Typically, the reduction of a carbonyl group to a methylene group with a catalyst B and H₂ in the presence of an additive is carried out as follows:

An anhydrous mixture of the sterically hindered amine ether (product of amine oxide process) and Pt/C or PtO₂ (0.0001-0.5 mole, preferably 0.001-0.3 mole per mole of sterically hindered amine ether moiety) in the presence of TiCl₄, AlCl₃ or ZrCl₄ (0.25-3 moles, preferably 0.5-1.5 moles per mole of sterically hindered amine ether moiety) in an organic solvent (alcohols like methanol, ethanol or iso-propanol, preferably iso-propanol; ethers like tetrahydrofuran, diethyl ether, t-butylmethyl ether; esters like ethyl acetate, methyl acetate, preferably ethyl acetate) is stirred at 0-100° C., preferably 20-80° C.; the hydrogen pressure is 1-120 bar, preferably 4-90 bar. Water is added and the reaction mixture extracted to afford the product.

Typically, the reduction of a carbonyl group to a methylene group with a hydrogen equivalent in the presence of an additive is carried out as follows:

A concentrated dichloromethane solution of the sterically hindered amine ether (product of amine oxide process) is added dropwise to chilled (e.g. −20 to 10° C., especially about 0° C.) dichloromethane through which a constant flow of BF₃ is passed. After the time necessary to form the BF₃-carbonyl group complex (e.g. 2-3 minutes), neat Et₃SiH (1-5 moles, preferably 1.2-4 moles, most preferably 1.5-3.5 moles per mole of sterically hindered amine ether moiety) is added rapidly to the solution. The reaction mixture is stirred at e.g. −20 to 10° C., especially about 0° C. until conversion is complete (e.g. 1-60 minutes). During addition of Et₃SiH and during the post-reaction slight bubbling of BF₃ through the reaction mixture is maintained. (The total amount of BF₃ is 1-5 moles, preferably 1.2-4 moles, most preferably 1.5-3.5 moles per mole of sterically hindered amine ether moiety). The product is isolated by quenching the reaction mixture with brine, separating-off the organic phase and evaporating the solvents.

Of interest is a sterically hindered amine ether of formula (A′) to (O′)

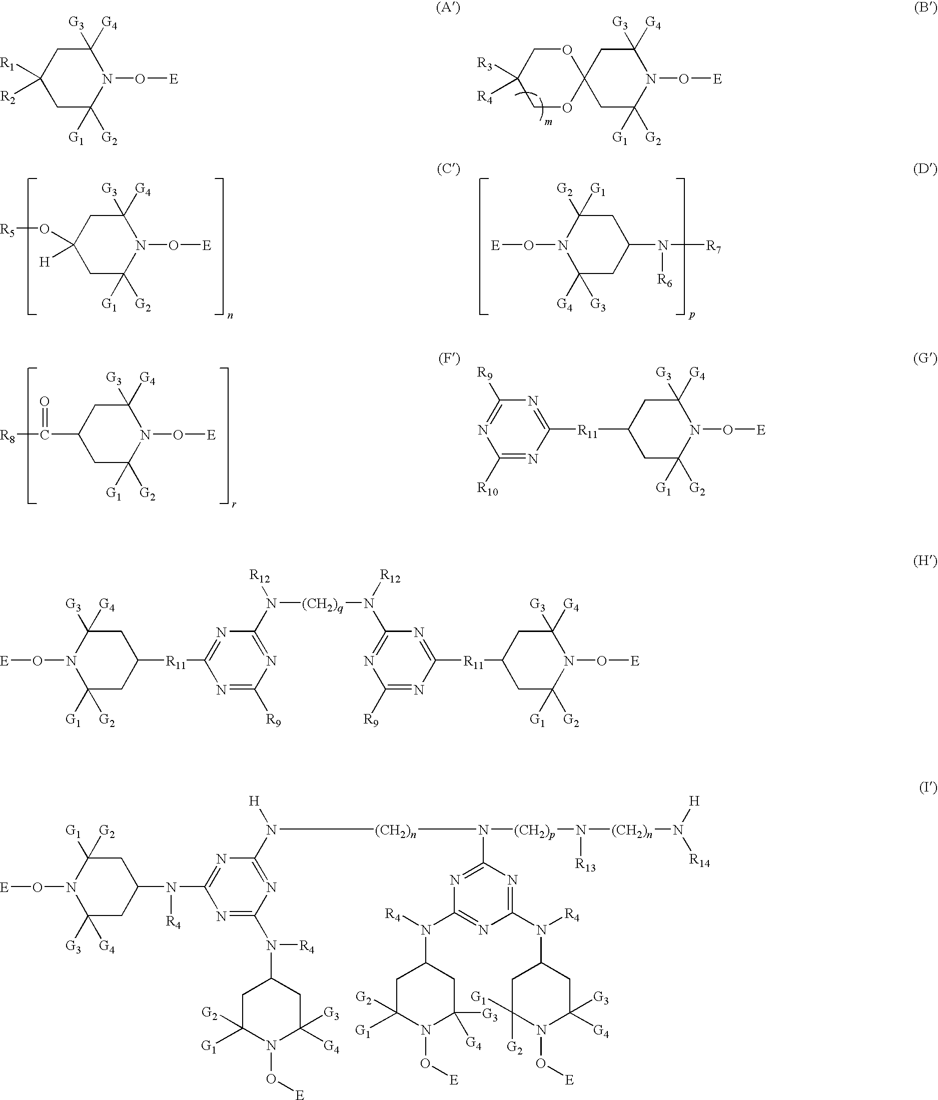

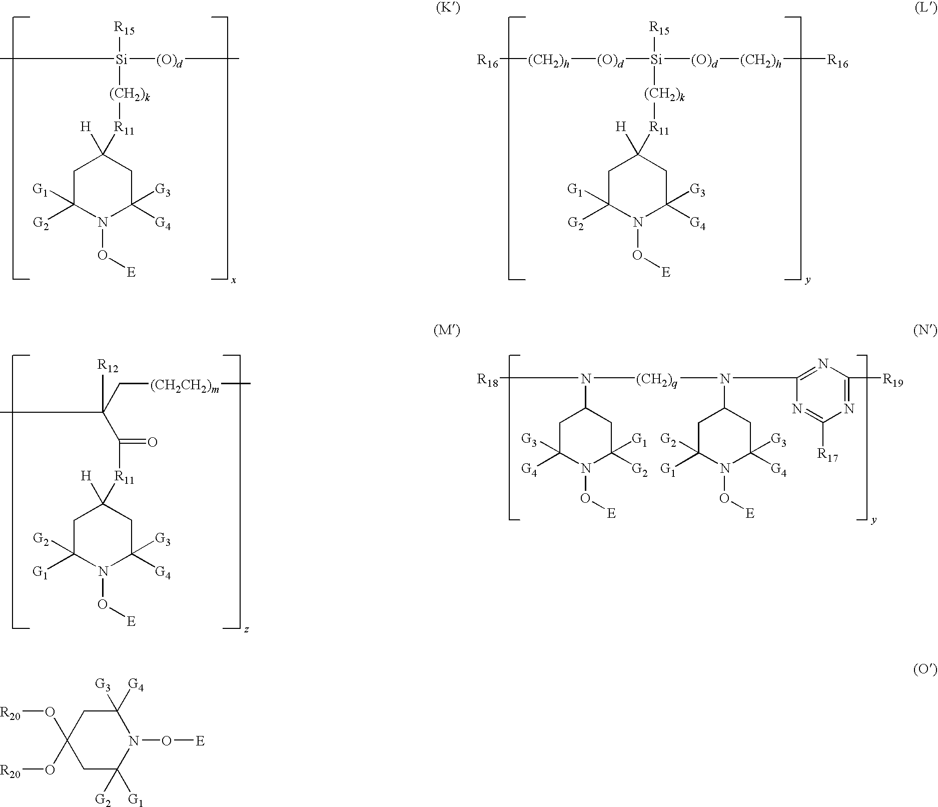

wherein G₁, G₂, G₃ and G₄ are as defined herein;

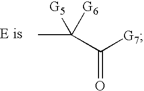

G₅, G₆ and G₇ are independently H, alkyl of 1 to 18 carbon atoms, alkenyl of 2 to 18 carbon atoms, alkinyl of 2 to 18 carbon atoms, aryl of 6 to 10 carbon atoms, aralkyl of 7 to 15 carbon atoms or COO(alkyl) of 2 to 19 carbon atoms; or
G₆ and G₇ form together alkylene of 3 to 10 carbon atoms;
R₁ and R₂ are together ═O;
m is 0 or 1;
R₃ is hydrogen, hydroxyl or hydroxymethyl;
R₄ is hydrogen, alkyl of 1 to 12 carbon atoms or alkenyl of 2 to 12 carbon atoms;
n is 1 to 4;
when n is 1,
R₅ is hydrogen, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms;
when n is 2,
R₅ is alkylene of 2 to 18 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when n is 3,
R₅ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
when n is 4,
R₅ is a tetravalent acyl radical of an aliphatic or unsaturated aliphatic tetracarboxylic acid, especially 1,2,3,4-butanetetracarboxylic acid, 1,2,3,4-but-2-enetetracarboxylic acid, 1,2,3,5-pentanetetracarboxylic acid and 1,2,4,5-pentanetetracarboxylic acid, or R₅ is a tetravalent acyl radical of an aromatic tetracarboxylic acid containing 10 to 18 carbon atoms;
p is 1 to 3;
R₆ is hydrogen, alkyl of 1 to 18 carbon atoms or acyl of 2 to 6 carbon atoms or phenyl;
when p is 1,
R₇ is hydrogen, phenyl, alkyl of 1 to 18 carbon atoms, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, an acyl radical of an aromatic carboxylic acid containing 7 to 15 carbon atoms, or R₆ and R₇ together are —(CH₂)₅CO—, phthaloyl or a divalent acyl radical of maleic acid;
when p is 2,
R₇ is alkylene of 2 to 12 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when p is 3,
R₇ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
r is 1 to 4;
when r is 1,
R₈ is alkoxy of 1 to 18 carbon atoms, alkenyloxy of 2 to 18 carbon atoms, —NHalkyl of 1 to 18 carbon atoms or —N(alkyl)₂ of 2 to 36 carbon atoms;
when r is 2,
R₈ is alkylenedioxy of 2 to 18 carbon atoms, alkenylenedioxy of 2 to 18 carbon atoms, —NH-alkylene-NH— of 2 to 18 carbon atoms or —N(alkyl)-alkylene-N(alkyl)- of 2 to 18 carbon atoms, or R₈ is 4-methyl-1,3-phenylenediamino;
when r is 3,
R₈ is a trivalent alkoxy radical of a saturated or unsaturated aliphatic triol containing 3 to 18 carbon atoms;
when r is 4,
R₈ is a tetravalent alkoxy radical of a saturated or unsaturated aliphatic tetraol containing 4 to 18 carbon atoms;
R₉ and R₁₀ are independently chlorine, alkoxy of 1 to 18 carbon atoms, —O-T₁, amino substituted by 2-hydroxyethyl, —NH(alkyl) of 1 to 18 carbon atoms, —N(alkyl)T₁ with alkyl of 1 to 18 carbon atoms, or —N(alkyl)₂ of 2 to 36 carbon atoms;
R₁₁ is oxygen, or R₁₁ is nitrogen substituted by either hydrogen, alkyl of 1 to 12 carbon atoms or T₁;

### T₁ is

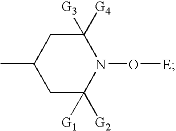

R₁₂ is hydrogen or methyl;
q is 2 to 8;
R₁₃ and R₁₄ are independently hydrogen or the group T₂;

### T₂ is

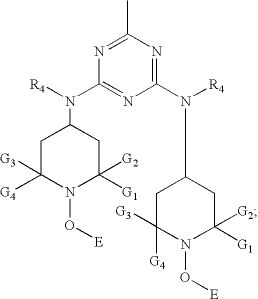

R₁₅ is hydrogen, phenyl, straight or branched alkyl of 1 to 12 carbon atoms, alkoxy of 1 to 12 carbon atoms, straight or branched alkyl of 1 to 4 carbon atoms substituted by phenyl, cycloalkyl of 5 to 8 carbon atoms, cycloalkenyl of 5 to 8 carbon atoms, alkenyl of 2 to 12 carbon atoms, glycidyl, allyloxy, straight or branched hydroxyalkyl of 1 to 4 carbon atoms, or silyl or silyloxy substituted three times independently by hydrogen, by phenyl, by alkyl of 1 to 4 carbon atoms or by alkoxy of 1 to 4 carbon atoms;
R₁₆ is hydrogen or silyl substituted three times independently by hydrogen, by phenyl, by alkyl of 1 to 4 carbon atoms or by alkoxy of 1 to 4 carbon atoms;
d is 0 or 1;
h is 0 to 4;
k is 0 to 5;
x is 3 to 6;
y is 1 to 10;
z is an integer such that the compound has a molecular weight of 1000 to 4000 amu, e.g. z may be from the range 3-10;
R₁₇ is morpholino, piperidino, 1-piperizinyl, alkylamino of 1 to 8 carbon atoms, especially branched alkylamino of 3 to 8 carbon atoms such as tert-octylamino, —N(alkyl)T₁ with alkyl of 1 to 8 carbon atoms, or —N(alkyl)₂ of 2 to 16 carbon atoms;
R₁₈ is hydrogen, acyl of 2 to 4 carbon atoms, carbamoyl substituted by alkyl of 1 to 4 carbon atoms, s-triazinyl substituted once by chlorine and once by R₁₇, or s-triazinyl substituted twice by R₁₇ with the condition that the two R₁₇ substituents may be different;
R₁₉ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃;

### T₃ is

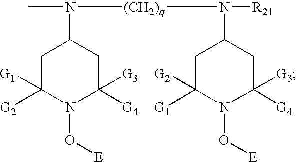

R₂₀ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms; and
R₂₁ is hydrogen, acyl of 2 to 4 carbon atoms, carbamoyl substituted by alkyl of 1 to 4 carbon atoms, s-triazinyl substituted twice by —N(alkyl)₂ of 2 to 16 carbon atoms or s-triazinyl substituted twice by —N(alkyl)T₁ with alkyl of 1 to 8 carbon atoms
with the proviso
that in formula (A′), G₇ is not methyl when G₅ and G₆ are H and
that in formula (C′), R₅ is not benzoyl when n is 1.

Preferably, in formula (A′), G₇ is not methyl when G₅ is H. More preferably, in formula (A′), G₇ is not methyl or ethyl when G₅ and G₆ are H. Even more preferably, in formula (A′), G₇ is not methyl or ethyl when G₅ is H. Most preferably, the sterically hindered amine ether is not of formula (A′).

Preferably, in formula (C′), when n is 1, R₅ is not an acyl radical of an aromatic acid containing 7 to 15 carbon atoms. More preferably, in formula (C′), when n is 1 and G₇ is methyl, R₅ is not an acyl radical of an aromatic acid containing 7 to 15 carbon atoms and G₅ and G₆ are not H. Even more preferably, in formula (C′), n is not 1. Most preferably, the sterically hindered amine ether is not of formula (C′).

Of special interest is a sterically hindered amine ether of formula (A′), (C′)-(F′), (I′) or (N′), in particular of formula (A′), (C′) or (N′).

Of more interest is a sterically hindered amine ether of formula (A′), (C′) or (N′), wherein

n is 1 to 2;
when n is 1,
R₅ is hydrogen or an acyl radical of an aliphatic or unsaturated aliphatic carboxylic or carbamic acid containing 2 to 18 carbon atoms;
when n is 2,
R₅ is alkylene of 2 to 18 carbon atoms, a divalent acyl radical of an aliphatic or unsaturated aliphatic dicarboxylic or dicarbamic acid containing 2 to 18 carbon atoms;
q is 4 to 8;
y is 2 to 10;
R₁₇ is alkylamino of 1 to 8 carbon atoms, especially branched alkylamino of 3 to 8 carbon atoms such as tert-octylamino, —N(alkyl)T₁ with alkyl of 1 to 8 carbon atoms, or —N(alkyl)₂ of 2 to 16 carbon atoms;
R₁₈ is hydrogen, s-triazinyl substituted once by chlorine and once by R₁₇, or s-triazinyl substituted twice by R₁₇ with the condition that the two R₁₇ substituents may be different;
R₁₉ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃;

### T₃ is

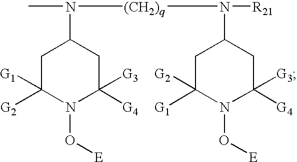

R₂₁ is hydrogen, s-triazinyl substituted twice by —N(alkyl)₂ of 2 to 16 carbon atoms or s-triazinyl substituted twice by —N(alkyl)T₁ with alkyl of 1 to 8 carbon atoms.

Of utmost interest is a sterically hindered amine ether of formula (A′) or (N′), wherein

q is 4 to 8;
y is 2 to 10;
R₁₇ is —N(alkyl)T₁ with alkyl of 1 to 8 carbon atoms, or —N(alkyl)₂ of 2 to 16 carbon atoms;
R₁₈ is s-triazinyl substituted twice by R₁₇;
R₁₉ is the group T₃;

### T₃ is

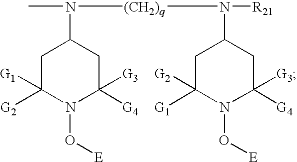

R₂₁ is s-triazinyl substituted twice by —N(alkyl)₂ of 2 to 16 carbon atoms.

For instance, the sterically hindered amine ether is of formula (N′), wherein

q is 6;
y is 2 to 10;
R₁₇ is —N(alkyl)T₁ with alkyl of 4 carbon atoms, or —N(alkyl)₂ of 8 carbon atoms;
R₁₈ is s-triazinyl substituted twice by R₁₇;
R₁₉ is the group T₃;

### T₃ is

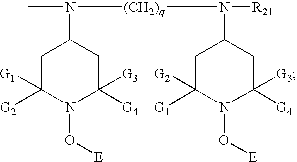

R₂₁ is s-triazinyl substituted twice by —N(alkyl)₂ of 8 carbon atoms.

An example is a sterically hindered amine ether, wherein

G₁, G₂, G₃ and G₄ are methyl.

Preferred is a sterically hindered amine ether, wherein

G₅, G₆ and G₇ are independently H, alkyl of 1 to 18 carbon atoms, alkenyl of 2 to 18 carbon atoms, or
G₆ and G₇ form together alkylene of 3 to 10 carbon atoms.

More preferred is a sterically hindered amine ether, wherein

G₅ and G₆ are independently H or as defined for G₇,
G₇ is alkyl of 1 to 8 carbon atoms or alkenyl of 2 to 8 carbon atoms, or
G₆ and G₇ form together alkylene of 3 to 6 carbon atoms.

Most preferred is a sterically hindered amine ether, wherein

G₅ and G₆ are independently H or methyl,
G₇ is alkyl of 1 to 3 carbon atoms or alkenyl of 2 to 4 carbon atoms, or
G₆ and G₇ form together alkylene of 4 carbon atoms.

For instance, E is

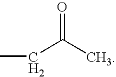

Of interest is a sterically hindered amine ether of formula (Z′)

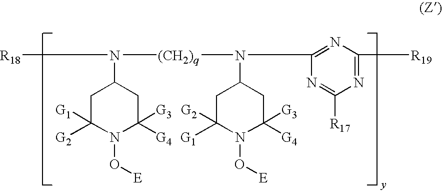

wherein E is

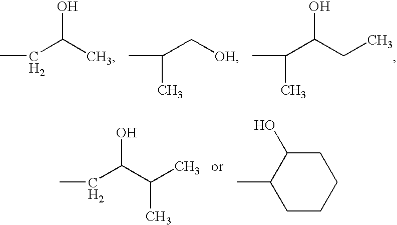

and
q, y, R₁₇, R₁₈ and R₁₉ are as defined above.

Preferences for the compound of formula (Z′) are analogously to the preferences for the compound of formula (N′).

In formula (Z′), E is preferably

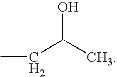

The instant compounds may be prepared according to one of the processes of this invention.

In the definitions the term alkyl comprises within the given limits of carbon atoms, for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, 2-methylheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl or dodecyl.

Examples of alkenyl are within the given limits of carbon atoms vinyl, allyl, and the branched and unbranched isomers of butenyl, pentenyl, hexenyl, heptenyl, octenyl, nonenyl, decenyl, undecenyl and dodecenyl. The term alkenyl also comprises residues with more than one double bond that may be conjugated or non-conjugated, for example may comprise one double bond.

Examples of alkinyl are within the given limits of carbon atoms ethinyl and propinyl and unbranched isomers of butinyl, pentinyl, hexinyl, heptinyl, octinyl, noninyl, decinyl, undecinyl and dodecinyl. The term alkinyl also comprises residues with more than one triple bond that may be conjugated or non-conjugated and residues with at least one triple bond and at least one double bond, for example comprises residues with one triple bond.

Examples of alkylene are within the given limits of carbon atoms branched and unbranched isomers of vinylene, allylene, butylene, pentylene, hexylene, heptylene, octylene, nonylene, decylene, undecylene and dodecylene.

Some examples of cycloalkyl are cyclopentyl, cyclohexyl, methylcyclopentyl, dimethylcyclopentyl and methylcyclohexyl.

Some examples of cycloalkenyl are cyclopentenyl, cyclohexenyl, methylcyclopentenyl, dimethylcyclopentenyl and methylcyclohexenyl. Cycloalkenyl may comprise more than one double bond that may be conjugated or non-conjugated, for example may comprise one double bond.

Aryl is for example phenyl or naphthyl.

Aralkyl is for instance benzyl or α,α-dimethylbenzyl.

The term alkoxy may comprise within the limits of the given number of carbon atoms, for example methoxy and ethoxy and the branched and unbranched isomers of propoxy, butoxy, pentyloxy, hexyloxy, heptyloxy, octyloxy, nonyloxy, decyloxy, undecyloxy, dodecyloxy, tridecyloxy, tetradecyloxy, pentadecyloxy, hexadecyloxy, heptadecyloxy and octadecyloxy.

The term halogen may comprise chlorine, bromine and iodine; for example halogen is chlorine.

The term halide may comprise fluoride, chloride, bromide or iodide.

For instance, alkali metal comprises Li, Na, K, Rb or Cs.

For example, alkaline-earth metal comprises Be, Mg, Ca, Sr or Ba.

Acyl radicals of monocarboxylic acids are, within the definitions, a residue of the formula —CO—R″, wherein R″ may stand inter alia for an alkyl, alkenyl, cycloalkyl or aryl radical as defined. Preferred acyl radicals include acetyl, benzoyl, acryloyl, methacryloyl, propionyl, butyryl, valeroyl, hexanoyl, heptanoyl, octanoyl, nonanoyl, decanoyl, undecanoyl, dodecanoyl, pentadecanoyl, stearoyl. Polyacyl radicals of polyvalent acids are of the formula (—CO)ₙ—R″, wherein n is the valency, e.g. 2, 3 or 4.

Some examples of an aliphatic carboxylic acid are acetic, propionic, butyric, stearic acid. An example of a cycloaliphatic carboxylic acid is cyclohexanoic acid. An example of an aromatic carboxylic acid is benzoic acid. An example of an aliphatic dicarboxylic acid is malonyl, maleoyl or succinyl, or sebacic acid. An example of a residue of an aromatic dicarboxylic acid is phthaloyl.

Of interest is a composition comprising

A) an organic polymer which is sensitive to oxidative, thermal and/or actinic degradation, and
B) at least one sterically hindered amine ether as defined above.

For instance, component (B) is present in an amount of 0.01-25% by weight of the organic polymer.

In general the instant sterically hindered amine ethers (component B) are added to the organic polymer to be stabilized in amounts of from 0.01 to 10%, preferably from 0.01 to 5%, in particular from 0.01 to 2% by weight of the organic polymer. Particular preference is given to the use of the instant sterically hindered amine ethers in amounts of from 0.05 to 1.5%, especially from 0.1 to 0.5% by weight of the organic polymer. Where the instant sterically hindered amine ethers are used as flame retardants, dosages are usually higher, e.g. 0.1 to 25%, mainly 0.1 to 10% by weight of the organic polymer.

Of interest are organic polymers that are natural, semi-synthetic or synthetic, especially a polyolefin or a polyolefin copolymer, for example a polyolefin.

Examples of polymers which can be protected with the compounds according to this invention are the following:

1. Polymers of monoolefins and diolefins, for example polypropylene, polyisobutylene, polybut-1-ene, poly-4-methylpent-1-ene, polyisoprene or polybutadiene, as well as polymers of cycloolefins, for instance of cyclopentene or norbornene, polyethylene (which optionally can be crosslinked), for example high density polyethylene (HDPE), high density and high molecular weight polyethylene (HDPE-HMW), high density and ultrahigh molecular weight polyethylene (HDPE-UHMW), medium density polyethylene (MDPE), low density polyethylene (LDPE), linear low density polyethylene (LLDPE), (VLDPE) and (ULDPE).

Polyolefins, i.e. the polymers of monoolefins exemplified in the preceding paragraph, preferably polyethylene and polypropylene, can be prepared by different, and especially by the following, methods:
a) radical polymerisation (normally under high pressure and at elevated temperature).
b) catalytic polymerisation using a catalyst that normally contains one or more than one metal of groups IVb, Vb, VIb or VIII of the Periodic Table. These metals usually have one or more than one ligand, typically oxides, halides, alcoholates, esters, ethers, amines, alkyls, alkenyls and/or aryls that may be either π- or σ-coordinated. These metal complexes may be in the free form or fixed on substrates, typically on activated magnesium chloride, titanium(III) chloride, alumina or silicon oxide. These catalysts may be soluble or insoluble in the polymerisation medium. The catalysts can be used by themselves in the polymerisation or further activators may be used, typically metal alkyls, metal hydrides, metal alkyl halides, metal alkyl oxides or metal alkyloxanes, said metals being elements of groups Ia, IIa and/or IIIa of the Periodic Table. The activators may be modified conveniently with further ester, ether, amine or silyl ether groups. These catalyst systems are usually termed Phillips, Standard Oil Indiana, Ziegler (-Natta), TNZ (DuPont), metallocene or single site catalysts (SSC).
   2. Mixtures of the polymers mentioned under 1), for example mixtures of polypropylene with polyisobutylene, polypropylene with polyethylene (for example PP/HDPE, PP/LDPE) and mixtures of different types of polyethylene (for example LDPE/HDPE).
   3. Copolymers of monoolefins and diolefins with each other or with other vinyl monomers, for example ethylene/propylene copolymers, linear low density polyethylene (LLDPE) and mixtures thereof with low density polyethylene (LDPE), propylene/but-1-ene copolymers, propylene/isobutylene copolymers, ethylene/but-1-ene copolymers, ethylene/hexene copolymers, ethylene/methylpentene copolymers, ethylene/heptene copolymers, ethylene/octene copolymers, propylene/butadiene copolymers, isobutylene/isoprene copolymers, ethylene/alkyl acrylate copolymers, ethylene/alkyl methacrylate copolymers, ethylene/vinyl acetate copolymers and their copolymers with carbon monoxide or ethylene/acrylic acid copolymers and their salts (ionomers) as well as terpolymers of ethylene with propylene and a diene such as hexadiene, dicyclopentadiene or ethylidene-norbornene; and mixtures of such copolymers with one another and with polymers mentioned in 1) above, for example polypropylene/ethylene-propylene copolymers, LDPE/ethylene-vinyl acetate copolymers (EVA), LDPE/ethylene-acrylic acid copolymers (EAA), LLDPE/EVA, LLDPE/EAA and alternating or random polyalkylene/carbon monoxide copolymers and mixtures thereof with other polymers, for example polyamides.
   4. Hydrocarbon resins (for example C₅-C₉) including hydrogenated modifications thereof (e.g. tackifiers) and mixtures of polyalkylenes and starch.
   5. Polystyrene, poly(p-methylstyrene), poly(α-methylstyrene).
   6. Copolymers of styrene or α-methylstyrene with dienes or acrylic derivatives, for example styrene/butadiene, styrene/acrylonitrile, styrene/alkyl methacrylate, styrene/butadiene/alkyl acrylate, styrene/butadiene/alkyl methacrylate, styrene/maleic anhydride, styrene/acrylonitrile/methyl acrylate; mixtures of high impact strength of styrene copolymers and another polymer, for example a polyacrylate, a diene polymer or an ethylene/propylene/diene terpolymer; and block copolymers of styrene such as styrene/butadiene/styrene, styrene/isoprene/styrene, styrene/ethylene/butylene/styrene or styrene/ethylene/propylene/styrene.
   7. Graft copolymers of styrene or α-methylstyrene, for example styrene on polybutadiene, styrene on polybutadiene-styrene or polybutadiene-acrylonitrile copolymers; styrene and acrylonitrile (or methacrylonitrile) on polybutadiene; styrene, acrylonitrile and methyl methacrylate on polybutadiene; styrene and maleic anhydride on polybutadiene; styrene, acrylonitrile and maleic anhydride or maleimide on polybutadiene; styrene and maleimide on polybutadiene; styrene and alkyl acrylates or methacrylates on polybutadiene; styrene and acrylonitrile on ethylene/propylene/diene terpolymers; styrene and acrylonitrile on polyalkyl acrylates or polyalkyl methacrylates, styrene and acrylonitrile on acrylate/butadiene copolymers, as well as mixtures thereof with the copolymers listed under 6), for example the copolymer mixtures known as ABS, MBS, ASA or AES polymers.
   8. Halogen-containing polymers such as polychloroprene, chlorinated rubbers, chlorinated and brominated copolymer of isobutylene-isoprene (halobutyl rubber), chlorinated or sulfo-chlorinated polyethylene, copolymers of ethylene and chlorinated ethylene, epichlorohydrin homo- and copolymers, especially polymers of halogen-containing vinyl compounds, for example polyvinyl chloride, polyvinylidene chloride, polyvinyl fluoride, polyvinylidene fluoride, as well as copolymers thereof such as vinyl chloride/vinylidene chloride, vinyl chloride/vinyl acetate or vinylidene chloride/vinyl acetate copolymers.
   9. Polymers derived from α,β-unsaturated acids and derivatives thereof such as polyacrylates and polymethacrylates; polymethyl methacrylates, polyacrylamides and polyacrylonitriles, impact-modified with butyl acrylate.
   10. Copolymers of the monomers mentioned under 9) with each other or with other unsaturated monomers, for example acrylonitrile/butadiene copolymers, acrylonitrile/alkyl acrylate copolymers, acrylonitrile/alkoxyalkyl acrylate or acrylonitrile/vinyl halide copolymers or acrylonitrile/alkyl methacrylate/butadiene terpolymers.
   11. Polymers derived from unsaturated alcohols and amines or the acyl derivatives or acetals thereof, for example polyvinyl alcohol, polyvinyl acetate, polyvinyl stearate, polyvinyl benzoate, polyvinyl maleate, polyvinyl butyral, polyallyl phthalate or polyallyl melamine; as well as their copolymers with olefins mentioned in 1) above.
   12. Homopolymers and copolymers of cyclic ethers such as polyalkylene glycols, polyethylene oxide, polypropylene oxide or copolymers thereof with bisglycidyl ethers.
   13. Polyacetals such as polyoxymethylene and those polyoxymethylenes which contain ethylene oxide as a comonomer; polyacetals modified with thermoplastic polyurethanes, acrylates or MBS.
   14. Polyphenylene oxides and sulfides, and mixtures of polyphenylene oxides with styrene polymers or polyamides.
   15. Polyurethanes derived from hydroxyl-terminated polyethers, polyesters or polybutadienes on the one hand and aliphatic or aromatic polyisocyanates on the other, as well as precursors thereof.
   16. Polyamides and copolyamides derived from diamines and dicarboxylic acids and/or from aminocarboxylic acids or the corresponding lactams, for example polyamide 4, polyamide 6, polyamide 6/6, 6/10, 6/9, 6/12, 416, 12/12, polyamide 11, polyamide 12, aromatic polyamides starting from m-xylene diamine and adipic acid; polyamides prepared from hexamethylenediamine and isophthalic or/and terephthalic acid and with or without an elastomer as modifier, for example poly-2,4,4-trimethylhexamethylene terephthalamide or poly-m-phenylene isophthalamide; and also block copolymers of the aforementioned polyamides with polyolefins, olefin copolymers, ionomers or chemically bonded or grafted elastomers; or with polyethers, e.g. with polyethylene glycol, polypropylene glycol or polytetramethylene glycol; as well as polyamides or copolyamides modified with EPDM or ABS; and polyamides condensed during processing (RIM polyamide systems).
   17. Polyureas, polyimides, polyamide-imides, polyetherimides, polyesterimids, polyhydantoins and polybenzimidazoles.
   18. Polyesters derived from dicarboxylic acids and diols and/or from hydroxycarboxylic acids or the corresponding lactones, for example polyethylene terephthalate, polybutylene terephthalate, poly-1,4-dimethylolcyclohexane terephthalate and polyhydroxybenzoates, as well as block copolyether esters derived from hydroxyl-terminated polyethers; and also polyesters modified with polycarbonates or MBS.
   19. Polycarbonates and polyester carbonates.
   20. Polysulfones, polyether sulfones and polyether ketones.
   21. Crosslinked polymers derived from aldehydes on the one hand and phenols, ureas and melamines on the other hand, such as phenol/formaldehyde resins, urea/formaldehyde resins and melamine/formaldehyde resins.
   22. Drying and non-drying alkyd resins.
   23. Unsaturated polyester resins derived from copolyesters of saturated and unsaturated dicarboxylic acids with polyhydric alcohols and vinyl compounds as crosslinking agents, and also halogen-containing modifications thereof of low flammability.
   24. Crosslinkable acrylic resins derived from substituted acrylates, for example epoxy acrylates, urethane acrylates or polyester acrylates.
   25. Alkyd resins, polyester resins and acrylate resins crosslinked with melamine resins, urea resins, isocyanates, isocyanurates, polyisocyanates or epoxy resins.
   26. Crosslinked epoxy resins derived from aliphatic, cycloaliphatic, heterocyclic or aromatic glycidyl compounds, e.g. products of diglycidyl ethers of bisphenol A and bisphenol F, which are crosslinked with customary hardeners such as anhydrides or amines, with or without accelerators.
   27. Blends of the aforementioned polymers (polyblends), for example PP/EPDM, Polyamide/EPDM or ABS, PVC/EVA, PVC/ABS, PVC/MBS, PC/ABS, PBTP/ABS, PC/ASA, PC/PBT, PVC/CPE, PVC/acrylates, POM/thermoplastic PUR, PC/thermoplastic PUR, POM/acrylate, POM/MBS, PPO/HIPS, PPO/PA 6.6 and copolymers, PA/HDPE, PA/PP, PA/PPO, PBT/PC/ABS or PBT/PET/PC.

Of particular interest is the use of compounds of formula (A′) to (O′) or (Z′) as stabilizers in synthetic organic polymers, for example a coating or a bulk polymer or article formed therefrom, especially in thermoplastic polymers and corresponding compositions as well as in coating compositions, for example in acid or metal catalyzed coating compositions. Thermoplastic polymers of most importance in present compositions are polyolefines (TPO) and their copolymers, such as listed above under items 1-3, thermoplastic polyurethan (TPU), thermoplastic rubber (TPR), polycarbonate, such as in item 19 above, and blends, such as in item 27 above. Of utmost importance are polyethylene (PE), polypropylene (PP), polycarbonate (PC) and polycarbonate blends such as PC/ABS blends.

Incorporation into the organic polymers can be effected, for example, by mixing in or applying the instant sterically hindered amine ethers and, if desired, further additives by the methods which are customary in the art. The incorporation can take place prior to or during the shaping operation, or by applying the dissolved or dispersed sterically hindered amine ethers to the polymer, with or without subsequent evaporation of the solvent. In the case of elastomers, these can also be stabilized as latices. A further possibility for incorporating the instant sterically hindered amine ethers into polymers is to add them before, during or directly after the polymerization of the corresponding monomers or prior to crosslinking. In this context the instant sterically hindered amine ethers can be added as it is or else in encapsulated form (for example in waxes, oils or polymers).

The instant sterically hindered amine ethers can also be added in the form of a masterbatch containing said compound in a concentration, for example, of from 2.5 to 25% by weight to the polymers that are to be stabilized.

The instant sterically hindered amine ethers can judiciously be incorporated by the following methods:
as emulsion or dispersion (e.g. to latices or emulsion polymers),
as a dry mixture during the mixing in of additional components or polymer mixtures,
by direct introduction into the processing apparatus (e.g. extruders, internal mixers, etc),
as solution or melt.

Novel polymer compositions can be employed in various forms and/or processed to give various products, for example as (to give) films, fibres, tapes, moulding compositions, profiles, or as binders for coating materials, adhesives or putties.

For example, the instant composition comprises further additives.

Examples of such further additives are phenolic antioxidants, aminic antioxidants, hindered amine light stabilizers, UV-absorbers, phosphites, phosphonites, benzofuranones, metal stearates, metal oxides, pigments, dyes, organophophorus compounds, hydroxylamines or flame retardants or mixtures thereof.

Examples of Further Additives are

1. Antioxidants

1.1. Alkylated monophenols, for example 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which are linear or branched in the side chains, for example, 2,6-di-nonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol and mixtures thereof.

1.2. Alkylthiomethylphenols, for example 2,4-dioctylthiomethyl-6-tert-butylphenol, 2,4-dioctylthiomethyl-6-methylphenol, 2,4-dioctylthiomethyl-6-ethylphenol, 2,6-di-dodecylthiomethyl-4-nonylphenol.

1.3. Hydroquinones and alkylated hydroquinones, for example 2,6-di-tert-butyl-4-methoxyphenol, 2,5-di-tert-butylhydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis-(3,5-di-tert-butyl-4-hydroxyphenyl) adipate.

1.4. Tocopherols, for example α-tocopherol, β-tocopherol, γ-tocopherol, δ-tocopherol and mixtures thereof (Vitamin E).

1.5. Hydroxylated thiodiphenyl ethers, for example 2, 2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis-(3,6-di-sec-amylphenol), 4,4′-bis(2,6-dimethyl-4-hydroxyphenyl)disulfide.

1.6. Alkylidenebisphenols, for example 2, 2′-methylenebis(6-tert-butyl-4-methylphenol), 2,2′-methylenebis(6-tert-butyl-4-ethylphenol), 2,2′-methylenebis[4-methyl-6-(α-methylcyclohexyl)-phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methyl-phenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis-(3,5-di-tert-butyl-4-hydroxyphenyl)propane, 2,2-bis-(5-tert-butyl-4-hydroxy-2-methylphenyl)-4-n-dodecylmercaptobutane, 1,1,5,5-tetra-(5-tert-butyl-4-hydroxy-2-methylphenyl)pentane.

1.7. O-, N- and S-benzyl compounds, for example 3, 5,3′,5′-tetra-tert-butyl-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide, isooctyl-3,5-di-tert-butyl-4-hydroxybenzylmercaptoacetate.

1.8. Hydroxybenzylated malonates, for example dioctadecyl-2,2-bis-(3,5-di-tert-butyl-2-hydroxybenzyl)-malonate, di-octadecyl-2-(3-tert-butyl-4-hydroxy-5-methylbenzyl)-malonate, di-dodecylmercaptoethyl-2,2-bis-(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate.

1.9. Aromatic hydroxybenzyl compounds, for example 1,3,5-tris-(3,5-di-tert-butyl-4-hydroxybenzyl)-2,4,6-trimethylbenzene, 1,4-bis(3,5-di-tert-butyl-4-hydroxybenzyl)-2,3,5,6-tetramethyl benzene, 2,4,6-tris(3,5-di-tert-butyl-4-hydroxybenzyl)phenol.

1.10. Triazine Compounds, for example 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris(3,5-di-tert-butyl-4-hydroxyphenoxy)-1,2,3-triazine, 1,3,5-tris-(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxyphenylethyl)-1,3,5-triazine, 1,3,5-tris(3,5-di-tert-butyl-4-hydroxyphenylpropionyl)-hexahydro-1,3,5-triazine, 1,3,5-tris(3,5-dicyclohexyl-4-hydroxybenzyl)isocyanurate.

1.11. Benzylphosphonates, for example dimethyl-2,5-di-tert-butyl-4-hydroxybenzylphosphonate, diethyl-3,5-di-tert-butyl-4-hydroxybenzylphosphonate, dioctadecyl-3,5-di-tert-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid.

1.12. Acylaminophenols, for example 4-hydroxylauranilide, 4-hydroxystearanilide, octyl N-(3,5-di-tert-butyl-4-hydroxyphenyl)carbamate.

1.13. Esters of β-(3,5-di-tert-butyl-4-hydroxyphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl) isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.14. Esters of 13-(5-tert-butyl-4-hydroxy-3-methylphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl) isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.15. Esters of β-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.16. Esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.17. Amides of β-(3,5-di-tert-butyl-4-hydroxyphenyl)propionic acid e.g. N,N′-bis(3,5-di-tert-butyl-4-hydroxyphenylpropionyl)hexamethylenediamide, N,N′-bis(3,5-di-tert-butyl-4-hydroxyphenylpropionyl)trimethylenediamide, N,N′-bis(3,5-di-tert-butyl-4-hydroxyphenylpropionyl)-hydrazide, N,N′-bis[2-(3-[3,5-di-tert-butyl-4-hydroxyphenyl]propionyloxy)ethyl]oxamide (Naugard® XL-1 supplied by Uniroyal).

1.18. Ascorbic acid (vitamin C)

1.19. Aminic antioxidants, for example N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine, 4-(p-toluenesulfamoyl)diphenylamine, N,N′-dimethyl-N,N′-di-sec-butyl-p-phenylenediamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octylated diphenylamine, for example p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis(4-methoxyphenyl)amine, 2,6-di-tert-butyl-4-dimethylaminomethylphenol, 2,4′-diaminodiphenylmethane, 4,4′-diaminodiphenylmethane, N,N,N′,N′-tetramethyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyldiphenylamines, a mixture of mono- und dialkylated tert-butyldiphenylamines, 2,3-dihydro-3,3-dimethyl-4H-1,4-benzothiazine, phenothiazine, a mixture of mono- und dialkylated tert-butyl/tert-octylphenothiazines, a mixture of mono- und dialkylated tert-octyl-phenothiazines, N-allylphenothiazin, N,N,N′,N′-tetraphenyl-1,4-diaminobut-2-ene, N,N-bis-(2,2,6,6-tetramethyl-piperid-4-yl-hexamethylenediamine, bis(2,2,6,6-tetramethylpiperid-4-yl)-sebacate, 2,2,6,6-tetramethylpiperidin-4-one, 2,2,6,6-tetramethylpiperidin-4-ol.

2. UV absorbers and light stabilisers

2.1. 2-(2′-Hydroxyphenyl)benzotriazoles, for example 2-(2′-hydroxy-5′-methylphenyl)-benzotriazole, 2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethyl butyl)phenyl)benzotriazole, 2-(3′,5′-di-tert-butyl-2′-hydroxyphenyl)-5-chloro-benzotriazole, 2-(3′-tert-butyl-2′-hydroxy-5′-methylphenyl)-5-chloro-benzotriazole, 2-(3′-sec-butyl-5′-tert-butyl-2′-hydroxyphenyl)benzotriazole, 2-(2′-hydroxy-4′-octyloxyphenyl)benzotriazole, 2-(3′,5′-di-tert-amyl-2′-hydroxyphenyl)benzotriazole, 2-(3′,5′-bis-(α,α-dimethyl benzyl)-2′-hydroxyphenyl)benzotriazole, 2-(3′-tert-butyl-2′-hydroxy-5′-(2-octyloxycarbonylethyl)phenyl)-5-chloro-benzotriazole, 2-(3′-tert-butyl-5′-[2-(2-ethylhexyloxy)-carbonylethyl]-2′-hydroxyphenyl)-5-chloro-benzotriazole, 2-(3′-tert-butyl-2′-hydroxy-5′-(2-methoxycarbonylethyl)phenyl)-5-chloro-benzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylene-bis-[4-(1,1,3,3-tetramethylbutyl)-6-benzotriazole-2-ylphenol]; the transesterification product of 2-[3′-tert-butyl-5′-(2-methoxycarbonylethyl)-2′-hydroxyphenyl]-2H-benzotriazole with polyethylene glycol 300; [R—CH₂CH₂—COO—CH₂CH₂₂ where R=3′-tert-butyl-4′-hydroxy-5-2H-benzotriazol-2-ylphenyl, 2-[2′-hydroxy-3′-(α,α-dimethyl benzyl)-5′-(1,1,3,3-tetramethylbutyl)-phenyl]benzotriazole; 2-[2′-hydroxy-3′-(1,1,3,3-tetramethylbutyl)-5′-(α,α-dimethyl benzyl)-phenyl]benzotriazole.

2.2. 2-Hydroxybenzophenones, for example the 4-hydroxy, 4-methoxy, 4-octyloxy, 4-decyloxy, 4-dodecyloxy, 4-benzyloxy, 4,2′,4′-trihydroxy and 2′-hydroxy-4,4′-dimethoxy derivatives.

2.3. Esters of substituted and unsubstituted benzoic acids, as for example 4-tertbutyl-phenyl salicylate, phenyl salicylate, octylphenyl salicylate, dibenzoyl resorcinol, bis(4-tert-butylbenzoyl) resorcinol, benzoyl resorcinol, 2,4-di-tert-butylphenyl 3,5-di-tert-butyl-4-hydroxybenzoate, hexadecyl 3,5-di-tert-butyl-4-hydroxybenzoate, octadecyl 3,5-di-tert-butyl-4-hydroxybenzoate, 2-methyl-4,6-di-tert-butylphenyl 3,5-di-tert-butyl-4-hydroxybenzoate.

2.4. Acrylates, for example ethyl α-cyano-β,β-diphenylacrylate, isooctyl α-cyano-β,β-diphenylacrylate, methyl α-carbomethoxycinnamate, methyl α-cyano-β-methyl-p-methoxy-cinnamate, butyl α-cyano-β-methyl-p-methoxy-cinnamate, methyl α-carbomethoxy-p-methoxycinnamate and N-(β-carbomethoxy-β-cyanovinyl)-2-methylindoline.

2.5. Nickel compounds, for example nickel complexes of 2,2′-thio-bis-[4-(1,1,3,3-tetramethylbutyl)phenol], such as the 1:1 or 1:2 complex, with or without additional ligands such as n-butylamine, triethanolamine or N-cyclohexyldiethanolamine, nickel dibutyldithiocarbamate, nickel salts of the monoalkyl esters, e.g. the methyl or ethyl ester, of 4-hydroxy-3,5-di-tert-butylbenzylphosphonic acid, nickel complexes of ketoximes, e.g. of 2-hydroxy-4-methylphenyl undecylketoxime, nickel complexes of 1-phenyl-4-lauroyl-5-hydroxypyrazole, with or without additional ligands.

2.6. Further sterically hindered amines, for example bis(2,2,6,6-tetramethyl-4-piperidyl)sebacate, bis(2,2,6,6-tetramethyl-4-piperidyl)succinate, bis(1,2,2,6,6-pentamethyl-4-piperidyl)sebacate, bis(1-octyloxy-2,2,6,6-tetramethyl-4-piperidyl)sebacate, bis(1,2,2,6,6-pentamethyl-4-piperidyl) n-butyl-3,5-di-tert-butyl-4-hydroxybenzylmalonate, the condensate of 1-(2-hydroxyethyl)-2,2,6,6-tetramethyl-4-hydroxypiperidine and succinic acid, linear or cyclic condensates of N,N′-bis(2,2,6,6-tetramethyl-4-piperidyl)hexamethylenediamine and 4-tert-octylamino-2,6-dichloro-1,3,5-triazine, tris(2,2,6,6-tetramethyl-4-piperidyl)nitrilotriacetate, tetrakis(2,2,6,6-tetramethyl-4-piperidyl)-1,2,3,4-butane-tetracarboxylate, 1,1′-(1,2-ethanediyl)-bis(3,3,5,5-tetra methyl piperazinone), 4-benzoyl-2,2,6,6-tetra methyl piperidine, 4-stearyloxy-2,2,6,6-tetramethylpiperidine, bis(1,2,2,6,6-pentamethylpiperidyl)-2-n-butyl-2-(2-hydroxy-3,5-di-tert-butylbenzyl)malonate, 3-n-octyl-7,7,9,9-tetramethyl-1,3,8-triazaspiro[4.5]decan-2,4-dione, bis(1-octyloxy-2,2,6,6-tetramethylpiperidyl)sebacate, bis(1-octyloxy-2,2,6,6-tetramethylpiperidyl)succinate, linear or cyclic condensates of N,N′-bis-(2,2,6,6-tetramethyl-4-piperidyl)hexamethylenediamine and 4-morpholino-2,6-dichloro-1,3,5-triazine, the condensate of 2-chloro-4,6-bis(4-n-butylamino-2,2,6,6-tetramethylpiperidyl)-1,3,5-triazine and 1,2-bis(3-aminopropylamino)ethane, the condensate of 2-chloro-4,6-di-(4-n-butylamino-1,2,2,6,6-pentamethylpiperidyl)-1,3,5-triazine and 1,2-bis-(3-aminopropylamino)ethane, 8-acetyl-3-dodecyl-7,7,9,9-tetramethyl-1,3,8-triazaspiro[4.5]decane-2,4-dione, 3-dodecyl-1-(2,2,6,6-tetramethyl-4-piperidyl)pyrrolidin-2,5-dione, 3-dodecyl-1-(1,2,2,6,6-pentamethyl-4-piperidyl)pyrrolidine-2,5-dione, a mixture of 4-hexadecyloxy- and 4-stearyloxy-2,2,6,6-tetramethylpiperidine, a condensation product of N,N′-bis(2,2,6,6-tetramethyl-4-piperidyl)hexamethylenediamine and 4-cyclohexylamino-2,6-dichloro-1,3,5-triazine, a condensation product of 1,2-bis(3-aminopropylamino)ethane and 2,4,6-trichloro-1,3,5-triazine as well as 4-butylamino-2,2,6,6-tetramethylpiperidine (CAS Reg. No. [136504-96-6]); N-(2,2,6,6-tetramethyl-4-piperidyl)-n-dodecylsuccinimid, N-(1,2,2,6,6-pentamethyl-4-piperidyl)-n-dodecylsuccinimid, 2-undecyl-7,7,9,9-tetramethyl-1-oxa-3,8-diaza-4-oxo-spiro[4,5]decane, a reaction product of 7,7,9,9-tetramethyl-2-cycloundecyl-1-oxa-3,8-diaza-4-oxospiro[4,5]decane und epichlorohydrin, 1,1-bis(1,2,2,6,6-pentamethyl-4-piperidyloxycarbonyl)-2-(4-methoxyphenyl)ethene, N,N′-bis-formyl-N,N′-bis(2,2,6,6-tetramethyl-4-piperidyl)hexamethylenediamine, diester of 4-methoxymethylene-malonic acid with 1,2,2,6,6-pentamethyl-4-hydroxypiperidine, poly[methylpropyl-3-oxy-4-(2,2,6,6-tetramethyl-4-piperidyl)]siloxane, reaction product of maleic acid anhydride-α-olefin-copolymer with 2,2,6,6-tetramethyl-4-aminopiperidine or 1,2,2,6,6-pentamethyl-4-aminopiperidine, 2,4-bis[N-(1-cyclohexyloxy-2,2,6,6-tetramethylpiperidine-4-yl)-N-butyl-amino]-6-(2-hydroxyethyl)amino-1,3,5-triazine.

2.7. Oxamides, for example 4, 4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide and its mixture with 2-ethoxy-2′-ethyl-5,4′-di-tert-butoxanilide, mixtures of o- and p-methoxy-disubstituted oxanilides and mixtures of o- and p-ethoxy-disubstituted oxanilides.

2.8. 2-(2-Hydroxyphenyl)-1,3,5-triazines, for example 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis-(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxy-propoxy)phenyl]-4,6-bis(2,4-dimethyl)-1,3,5-triazine, 2-[2-hydroxy-4-(2-hydroxy-3-octyloxy-propyloxy)phenyl]-4,6-bis(2,4-dimethyl)-1,3,5-triazine, 2-[4-(dodecyloxy/tridecyloxy-2-hydroxypropoxy)-2-hydroxy-phenyl]-4,6-bis(2,4-di-methylphenyl)-1,3,5-triazine, 2-[2-hydroxy-4-(2-hydroxy-3-dodecyloxy-propoxy)phenyl]-4,6-bis(2,4-dimethylphenyl)-1,3,5-triazine, 2-(2-hydroxy-4-hexyloxy)phenyl-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxy-propoxy)phenyl]-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxypropyloxy]phenyl}-4,6-bis(2,4-dimethylphenyl)-1,3,5-triazine, 2-{2-hydroxy-4-[1-octyloxycarbonyl-ethoxy]phenyl}-4,6-bis(4-phenylphenyl)-1,3,5-triazine wherein the octyl moiety is a mixture of different isomers.

3. Metal deactivators, for example N,N′-diphenyloxamide, N-salicylal-N′-salicyloyl hydrazine, N,N′-bis(salicyloyl) hydrazine, N,N′-bis(3,5-di-tert-butyl-4-hydroxyphenylpropionyl) hydrazine, 3-salicyloylamino-1,2,4-triazole, bis(benzylidene)oxalyl dihydrazide, oxanilide, isophthaloyl dihydrazide, sebacoyl bisphenylhydrazide, N,N′-diacetyladipoyl dihydrazide, N,N′-bis(salicyloyl)oxalyl dihydrazide, N,N′-bis(salicyloyl)thiopropionyl dihydrazide.

4. Phosphites and phosphonites, for example triphenyl phosphite, diphenyl alkyl phosphites, phenyl dialkyl phosphites, tris(nonylphenyl) phosphite, trilauryl phosphite, trioctadecyl phosphite, distearyl pentaerythritol diphosphite, tris(2,4-di-tert-butylphenyl) phosphite, diisodecyl pentaerythritol diphosphite, bis(2,4-di-tert-butylphenyl) pentaerythritol diphosphite, bis(2,6-di-tert-butyl-4-methylphenyl)-pentaerythritol diphosphite, diisodecyloxypentaerythritol diphosphite, bis(2,4-di-tert-butyl-6-methylphenyl)pentaerythritol diphosphite, bis(2,4,6-tris(tert-butylphenyl)pentaerythritol diphosphite, tristearyl sorbitol triphosphite, tetrakis(2,4-di-tert-butylphenyl) 4,4′-biphenylene diphosphonite, 6-isooctyloxy-2,4,8,10-tetra-tert-butyl-12H-dibenz[d,g]-1,3,2-dioxaphosphocin, 6-fluoro-2,4,8,10-tetra-tert-butyl-12-methyl-dibenz[d,g]-1,3,2-dioxaphosphocin, bis(2,4-di-tert-butyl-6-methylphenyl)methyl phosphite, bis(2,4-di-tert-butyl-6-methylphenyl)ethyl phosphite, 2,2′,2″-nitrilo[triethyltris(3,3′,5,5′-tetra-tert-butyl-1,1′-biphenyl-2,2′-diyl)phosphite], 2-ethylhexyl(3,3′,5,5′-tetra-tert-butyl-1,1′-biphenyl-2,2′-diyl)phosphite.

Especially preferred are the following phosphites:

Tris(2,4-di-tert-butylphenyl) phosphite (Irgafos® 168, Ciba Specialty Chemicals), tris(nonylphenyl) phosphite,

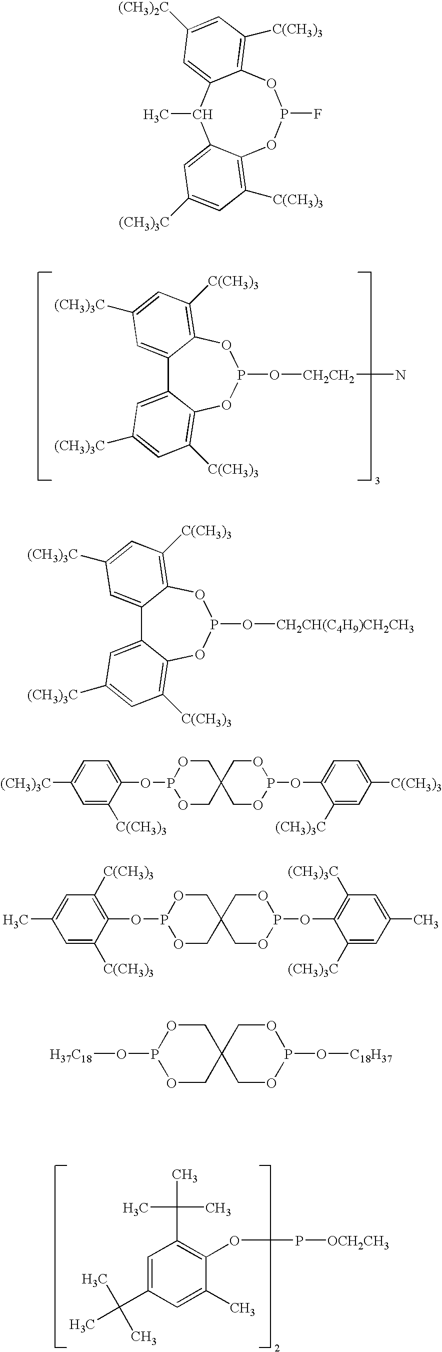

5. Hydroxylamines, for example, N,N-dibenzylhydroxylamine, N,N-diethylhydroxylamine, N,N-dioctylhydroxylamine, N,N-dilaurylhydroxylamine, N,N-ditetradecylhydroxylamine, N,N-dihexadecylhydroxylamine, N,N-dioctadecylhydroxylamine, N-hexadecyl-N-octadecylhydroxylamine, N-heptadecyl-N-octadecylhydroxylamine, N,N-dialkylhydroxylamine derived from hydrogenated tallow amine.

6. Nitrones, for example, N-benzyl-alpha-phenyl-nitrone, N-ethyl-alpha-methyl-nitrone, N-octyl-alpha-heptyl-nitrone, N-lauryl-alpha-undecyl-nitrone, N-tetradecyl-alpha-tridcyl-nitrone, N-hexadecyl-alpha-pentadecyl-nitrone, N-octadecyl-alpha-heptadecyl-nitrone, N-hexadecyl-alpha-heptadecyl-nitrone, N-ocatadecyl-alpha-pentadecyl-nitrone, N-heptadecyl-alpha-heptadecyl-nitrone, N-octadecyl-alpha-hexadecyl-nitrone, nitrone derived from N,N-dialkylhydroxylamine derived from hydrogenated tallow amine.

7. Thiosynergists, for example, dilauryl thiodipropionate or distearyl thiodipropionate.

8. Peroxide scavengers, for example esters of β-thiodipropionic acid, for example the lauryl, stearyl, myristyl or tridecyl esters, mercaptobenzimidazole or the zinc salt of 2-mercaptobenzimidazole, zinc dibutyldithiocarbamate, dioctadecyl disulfide, pentaerythritol tetrakis(β-dodecylmercapto)propionate.

9. Polyamide stabilisers, for example, copper salts in combination with iodides and/or phosphorus compounds and salts of divalent manganese.

10. Basic co-stabilisers, for example, melamine, polyvinylpyrrolidone, dicyandiamide, triallyl cyanurate, urea derivatives, hydrazine derivatives, amines, polyamides, polyurethanes, alkali metal salts and alkaline earth metal salts of higher fatty acids for example calcium stearate, zinc stearate, magnesium behenate, magnesium stearate, sodium ricinoleate and potassium palmitate, antimony pyrocatecholate or zink pyrocatecholate.

11. Nucleating agents, for example, inorganic substances such as talcum, metal oxides such as titanium dioxide or magnesium oxide, phosphates, carbonates or sulfates of, preferably, alkaline earth metals; organic compounds such as mono- or polycarboxylic acids and the salts thereof, e.g. 4-tert-butylbenzoic acid, adipic acid, diphenylacetic acid, sodium succinate or sodium benzoate; polymeric compounds such as ionic copolymers (ionomers).

12. Fillers and reinforcing agents, for example, calcium carbonate, silicates, glass fibres, glass bulbs, asbestos, talc, kaolin, mica, barium sulfate, metal oxides and hydroxides, carbon black, graphite, wood flour and flours or fibers of other natural products, synthetic fibers.

13. Other additives, for example, plasticisers, lubricants, emulsifiers, pigments, rheology additives, catalysts, flow-control agents, optical brighteners, flameproofing agents, antistatic agents and blowing agents.

14. Benzofuranones and indolinones, for example those disclosed in U.S. Pat. No. 4,325,863; U.S. Pat. No. 4,338,244; U.S. Pat. No. 5,175,312; U.S. Pat. No. 5,216,052; U.S. Pat. No. 5,252,643; DE-A-4316611; DE-A-4316622; DE-A-4316876; EP-A-0589839 or EP-A-0591102 or 3-[4-(2-acetoxyethoxy)-phenyl]-5,7-di-tert-butyl-benzofuran-2-one, 5,7-di-tert-butyl-3-[4-(2-stearoyloxyethoxy)phenyl]benzofuran-2-one, 3,3′-bis[5,7-di-tert-butyl-3-(4-[2-hydroxyethoxy]phenyl)benzofuran-2-one], 5,7-di-tert-butyl-3-(4-ethoxyphenyl)benzofuran-2-one, 3-(4-acetoxy-3,5-dimethylphenyl)-5,7-di-tert-butyl-benzofuran-2-one, 3-(3,5-dimethyl-4-pivaloyloxyphenyl)-5,7-di-tert-butyl-benzofuran-2-one, 3-(3,4-dimethylphenyl)-5,7-di-tert-butyl-benzofuran-2-one, 3-(2,3-dimethylphenyl)-5,7-di-tert-butyl-benzofuran-2-one.

The conventional additives are judiciously employed in amounts of 0.1-10% by weight, for example 0.2-5% by weight, based on the organic polymer.

Of interest is a process for flame retarding an organic polymer or stabilizing an organic polymer against degradation by light, oxygen and/or heat, which process comprises applying to or incorporating into said polymer at least one sterically hindered amine ether as defined above.

Also of interest is the use of at least one sterically hindered amine ether as defined above as a stabilizer for an organic polymer against degradation by light, oxygen and/or heat or as flame retardant for an organic polymer.

All percentages and ratios given are weight percentages and weight ratios unless otherwise stated.

### ABBREVIATIONS AND FORMULA

comp. compound
eq. equivalent
GLC gas liquid chromatography
TEMPO 2,2,6,6-tetramethylpiperidine-N-oxide
THF tetrahydrofuran

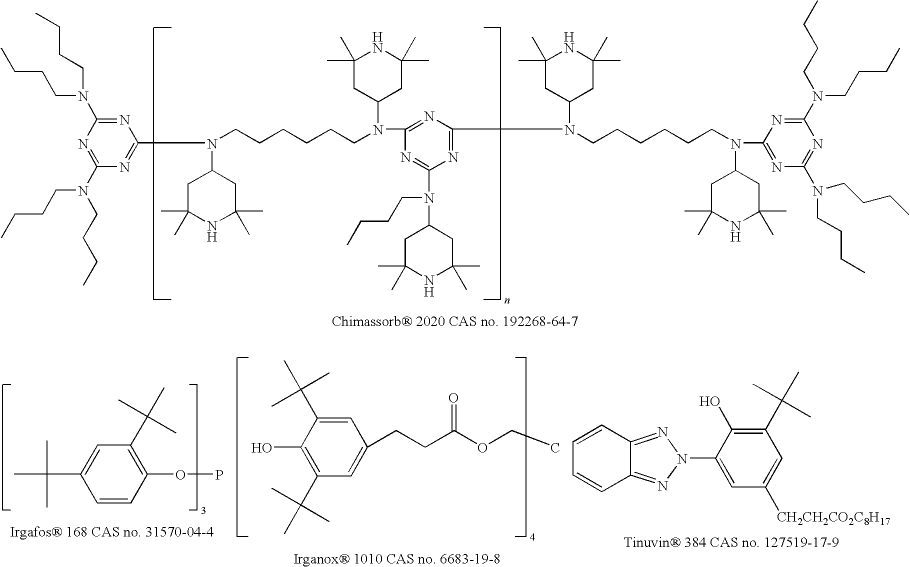

### COMPOUND EXAMPLES

### Example 1

### Preparation of Compound A1 Using AgNO₃ as Catalyst

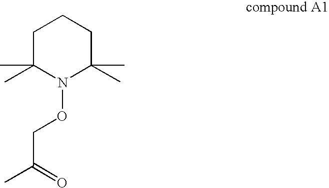

To a stirred solution of 0.17 g (1 mmol) AgNO₃ in 30 ml deionized water is added at 25° C. a solution of 7.8 g (50 mmol) TEMPO in 30.5 g (525 mmol) acetone. After the mixture is brought to reflux (ca. 62° C.) a solution of 11.9 g (50 mmol) Na₂S₂O₈ in 45 ml water is slowly dosed in until the initial red colour disappears (after 30 ml/3 hrs). The mixture is cooled down to 25° C. and the organic phase split off. The aqueous phase is neutralized with NaHCO₃, saturated with NaCl and extracted with pentane. The combined organic phases are washed with brine and concentrated on a rotary evaporator to yield 8.7 g (82%) of a liquid. Kugelrohr distillation at 80° C./0.5 mbar affords 7 g (66%) of a liquid.

Anal. Calculated for C₁₂H₂₃NO₂ (213.32): C, 67.57; H, 10.87; N, 6.57. Found: C, 67.60; H, 10.74; N, 6.45.

¹H-NMR (400 MHz, CDCl₃), δ (ppm): 0.9-1.2 (m, 12H), 1.25-1.7 (m, 6H), 2.21 (s, 3H), 4.38 (s, 2H).

### Example 2

### Preparation of Compound A1 without Catalyst

To a stirred solution of 5.5 g (35 mmol) TEMPO in 21.4 g (368 mmol) acetone is slowly added at 60° C. an aqueous solution of 8.3 g (35 mmol) Na₂S₂O₈ dissolved in 30 ml water until disappearance of the initial red color (after 20 ml/ca 3 hours). The aqueous phase is neutralized with NaHCO₃, saturated with NaCl and the reaction mixture extracted with hexane. The combined organic phases are washed with brine and concentrated on a rotary evaporator to yield 5.3 g (72%) of a liquid exhibiting the same ¹H-NMR as in Example 1.

¹³C-NMR (100 MHz, CDCl₃), δ (ppm): 16.94, 20.10, 27.19, 32.85, 39.59, 60.00, 83.28 and 206.68.

### Example 3

### Preparation of Compound A1 without Catalyst, but in Presence of NaHCO₃

To a stirred mixture of 5.5 g (35 mmol) TEMPO, 21.4 g (368 mmol) acetone and 5.9 g (70 mmol) NaHCO₃ is slowly added at 60° C. an aqueous solution of 8.3 g (35 mmol) Na₂S₂O₈ dissolved in 30 ml water until disappearance of the initial red color (ca 8 hours). The organic phase is split off and brine-washed. The aqueous phase (pH 7-8) is extracted with hexane. The combined organic phases are dried over MgSO₄ and concentrated on a rotary evaporator to yield 6.4 g (86%) of a liquid exhibiting the same ¹H-NMR as in Example 1.

Anal. Calculated for C₁₂H₂₃NO₂ (213.32): C, 67.57; H, 10.87; N, 6.57. Found: C, 67.78; H, 11.33; N, 6.61.

### Example 4

### Preparation of Compound A1 without Catalyst, but in Presence of Na₂CO₃

To a stirred mixture of 5.5 g (35 mmol) TEMPO, 21.4 g (368 mmol) acetone and 3.7 g (34.9 mmol) Na₂CO₃ is slowly added at 60° C. an aqueous solution of 8.3 g (35 mmol) Na₂S₂O₈ dissolved in 30 ml water until disappearance of the initial red color (ca 6 hours). The organic phase is split off and brine-washed. The aqueous phase (pH 7-8) is extracted with hexane. The combined organic phases are dried over MgSO₄ and concentrated on a rotary evaporator to yield 5.4 g (72%) of a liquid exhibiting the same ¹H-NMR as in Example 1.

Anal. Calculated for C₁₂H₂₃NO₂ (213.32): C, 67.57; H, 10.87; N, 6.57. Found: C, 67.53; H, 10.73; N, 6.42.

### Example 5

### Preparation of Compound A2

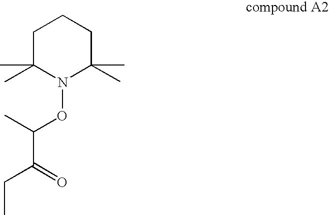

Prepared using 0.17 g (1 mmol, dissolved in 1 ml water) AgNO₃, 7.8 g (50 mmol) TEMPO, 43.1 g (500 mmol) 3-pentanone and 11.9 g (50 mmol, dissolved in 40 ml water) Na₂S₂O₈ similar to Example 1, except that all of the Na₂S₂O₈ is added (duration 4.3 hrs). Yield (crude): 9.2 g (76%) of a liquid.

Anal. Calculated for C₁₄H₂₇NO₂ (241.38): C, 69.67; H, 11.28; N, 5.80. Found: C, 68.62; H, 11.05; N, 5.77.

¹H-NMR (400 MHz, CDCl₃), δ (ppm): 1.0-1.5 (m, 24H), 2.52-2.60 (m, 1H), 2.67-2.75 (m, 1H), 4.30 (q, 1H).

### Example 6

### Preparation of Compound A3

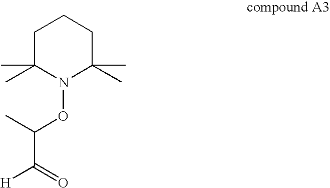

Prepared using 0.17 g (1 mmol, neat) AgNO₃, 7.8 g (50 mmol) TEMPO, 29.6 g (500 mmol) propionaldehyde and 11.9 g (50 mmol, dissolved in 40 ml water) Na₂S₂O₈ similar to Example 1, except that only 60% of the Na₂S₂O₈ is added (duration 3.8 hrs). The crude product is purified by flash chromatography using silica gel and hexane/ethylacetate 9/1. Yield: 3.2 g (30%) of a liquid.

Anal. Calculated for C₁₂H₂₃NO₂ (213.32): C, 67.57; H, 10.87; N, 6.57. Found: C, 67.78; H, 10.77; N, 6.23.

¹H-NMR (400 MHz, CDCl₃), δ (ppm): 1.0-1.6 (m, 21H), 4.16-4.22 (m, 1H), 9.76 (d, 1H).

### Example 7

### Preparation of Compound A4

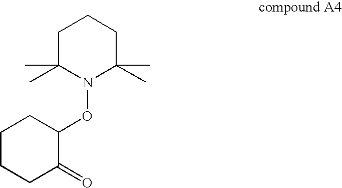

Prepared using 0.17 g (1 mmol, dissolved in 1 ml water) AgNO₃, 7.8 g (50 mmol) TEMPO, 49.1 g (500 mmol) cyclohexanone and 11.9 g (50 mmol, dissolved in 40 ml water) Na₂S₂O₈ similar to Example 1, except that only 50% of the Na₂S₂O₈ is added (duration 3.2 hrs) and that the organic phase is washed with sodium thiosulphate (Na₂S₂O₃) to destroy residual peroxide. The crude product is purified by flash chromatography using silica gel and hexane/ethylacetate 9/1. Yield: 4.4 g (35%) of a liquid.

Anal. Calculated for C₁₅H₂₇NO₂ (253.39): C, 71.10; H, 10.74; N, 5.53. Found: C, 71.12; H, 10.64; N, 5.51.

¹H-NMR (300 MHz, CDCl₃), δ (ppm): 1.0-2.2 (m, 24H), 2.21-2.3 (m, 1H), 2.7-2.8 (m, 1H), 4.14-4.18 (m, 1H).

### Example 8

### Preparation of Compound A5

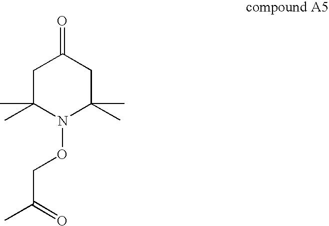

Prepared using 0.17 g (1 mmol, dissolved in 20 ml water) AgNO₃, 8.5 g (50 mmol) triacetoneamine-N-oxide, 30.5 g (500 mmol) acetone and 11.9 g (50 mmol, dissolved in 40 ml water) Na₂S₂O₈ similar to Example 1, except that only 60% of the Na₂S₂O₈ is added (duration 7 hrs). Part of the crude product (80%, 5 g) is purified by flash chromatography using silica gel and hexane/ethylacetate 8/2. Yield: 1.8 g (16%) of crystals.

Anal. Calculated for C₁₂H₂₁NO₃ (227.31): C, 63.41; H, 9.31; N, 6.16. Found: C, 63.33; H, 9.30; N, 6.07.

¹H-NMR (400 MHz, CDCl₃), δ (ppm): 1.22 (s, 6H), 1.30 (s, 6H), 2.21 (s, 3H), 2.22 (d-like, 2H), 2.59 (d-like, 2H), 4.50 (s, 2H).

### Example 9

### Preparation of Compounds A6 and A7

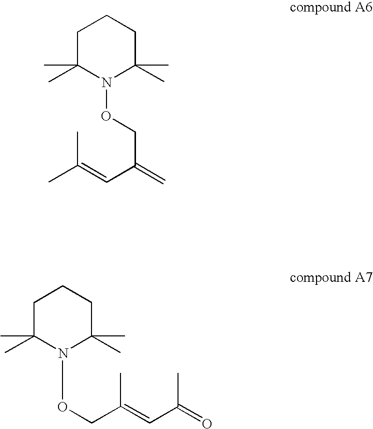

Prepared using 0.17 g (1 mmol, dissolved in 1 ml water) AgNO₃, 7.8 g (50 mmol) TEMPO, 54.5 g (500 mmol) mesityl oxide and 11.9 g (50 mmol, dissolved in 40 ml water) Na₂S₂O₈ similar to Example 1, except that all of the Na₂S₂O₈ is added (duration 4.5 hrs). Yield (crude): 9.14 g (72%) of a liquid, a mixture of the compounds A6 and A7 (about 1:1 by ¹H-NMR).

LC/MS (m/z): 254 (MH⁺; C₁₅H₂₇NO₂, 253.39; 2 isomers)

¹H-NMR (500 MHz, CDCl₃), δ (ppm):

compound A6: 1.14 (s, 6H), 1.17 (s, 6H), 1.25-1.65 (m, 6H), 1.94 (d, 3H), 2.20 (d, 3H), 4.40 (s, 2H), 6.30 (p-like, 1H).

compound A7: 1.15 (s, 12H), 1.30-1.65 (m, 6H), 2.03 (m, 3H), 2.23 (s, 3H), 4.29 (d-like, 2H), 6.38 (m, 1H); trans configuration established by means of NOE (═CCH₂O cis to ═CH due to NOE; ═CCH₃ trans to ═CH due to absence of NOE).

### Example 10

### Preparation of Compounds A8 and A9

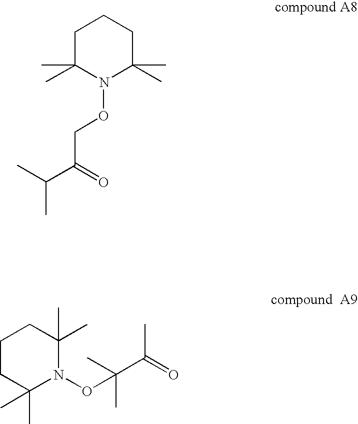

Prepared using 0.17 g (1 mmol, dissolved in 1 ml water) AgNO₃, 7.8 g (50 mmol) TEMPO, 43.2 g (500 mmol) 3-methyl-2-butanone and 12 g (50 mmol, dissolved in 40 ml water) Na₂S₂O₈ similar to Example 1, except that all of the Na₂S₂O₈ is added (duration 4.5 hrs). The crude product is purified by flash chromatography using silica gel and hexane/ethylacetate 9/1. Yield: 5 g (41%) of a liquid, a mixture of the compounds A8 and A9 (about 1:1 by ¹H-NMR).

LC/MS (m/z): 242 (MH⁺; C₁₄H₂₇NO₂, 241.38; 2 isomers)

¹H-NMR (300 MHz, CDCl₃), δ (ppm):

compound A8: 1.11 (s, 6H), 1.13 (d, 6H), 1.16 (s, 6H), 1.25-1.65 (m, 6H), 2.83 (p, 1H), 4.50 (s, 2H).

compound A9: 0.99 (s, 6H), 1.14 (s, 6H), 1.25-1.65 (m, 12H), 2.29 (s, 3H).

### Example 11

### Preparation of Compound A10

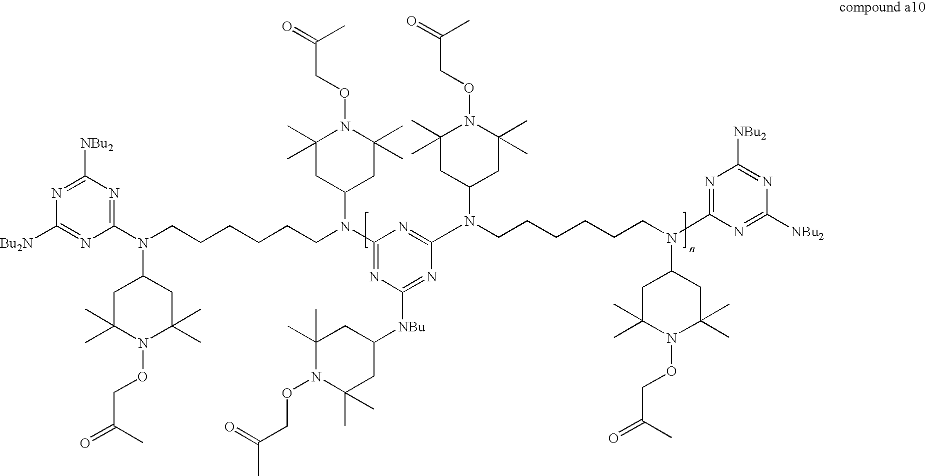

### Step 1:

To a mixture of 20 g Chimassorb® 2020 (Mₙ by GPC: 2819 g/mole, ca. 3.5 meq NH/g) and 35.5 g (336.5 mmol) Na₂CO₃ in 40 ml CH₂Cl₂ are slowly added at −5° C. 26.6 g (136.5 mmol) AcOOH (39% in AcOH). The mixture is kept stirable by concomitant, slow addition of a total of 90 ml water. The mixture is then stirred overnight at 20° C. and the organic phase split off. The aqueous phase is extracted with CH₂Cl₂ and the combined organic phases washed with NaOH and brine, dried over MgSO₄ and the solvent evaporated to afford 18 g of a powder.

Anal. Found: C, 65.17; H, 10.00; N, 16.84; 0, 6.64.

### Step 2:

5.2 g (ca 3.3 meq NO/g) of the product of step 1 are dissolved in 10.7 g (184 mmol) acetone. The red solution is stirred and brought to 60° C. followed by the addition of 4.2 g (17.6 mmol) Na₂S₂O₈, dissolved in 15 ml water, within 3 hours. The solution is kept at 60° C. for 1.5 hours, after which the color turns from red to orange. An additional 2.1 g (8.8 mmol) Na₂S₂O₈, dissolved in 7.5 ml water, are added within 1 hour. The mixture is stirred at 60° C. for 0.5 hours, after which the color turns from orange to slightly yellow. The mixture is neutralized (NaHCO₃), extracted with CH₂Cl₂ and the organic phase brine-washed and dried over MgSO₄. Evaporation of the solvent yields 4.7 g of a powder.

Anal. Found: C, 64.59; H, 9.76; N, 14.22; 0, 9.88.

¹H-NMR (400 MHz, CDCl₃), δ (ppm, NO—CH₂COCH₃ only): 4.42 (br s)

### Example 12

### Carbonyl to Methylene Reduction of Compound A1 Using H₂/Catalyst/Additive

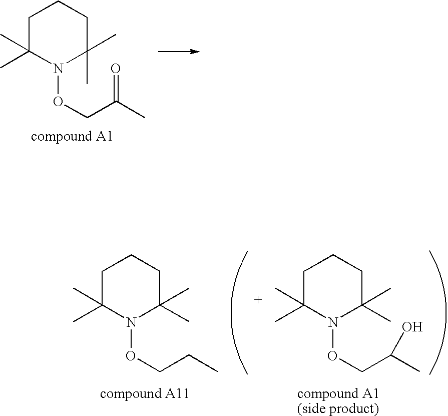

Method 1: In a glass vial, the (anhydrous) catalyst (amount relative to compound A1) and the additive (equivalents relative to compound A1) are added under argon. Thereafter, 2 ml solvent and 100 mg (0.47 mmol) compound A1 are added. The glass vial is transferred into an autoclave, the system sealed and flushed with argon (3×) and hydrogen (3×). The autoclave is tested for tightness and the reaction mixture stirred overnight at 6 bar hydrogen pressure/25° C. The autoclave is then flushed with argon, the reaction mixture filtered and the solvent evaporated. The residue is extracted with dichloromethane, water-washed and analyzed by GLC (area %). Compounds A11 and A12 are identified by means of their retention times, which have been previously determined using independently synthesized samples (compound A11: according to DE19907945; compound A12: Example 13). The catalyst (5% Pt/C) is from Engelhard.

| | | | | | Comp. | Comp. | Comp. | |
|---|---|---|---|---|---|---|---|---|
| | Catalyst | Catalyst | Additive | | A1 | A12 | A11 | Unknown |
| Experiment | (% w/w) | (mol. eq.) | (mol. eq.) | Solvent | (area %) | (area %) | (area %) | (area %) |
| A | PtO₂ (30) | 0.282 | AlCl₃ (2.6) | EtOAc | 9 | 0 | 86 | 5 |
| B | Pt/C (50) | 0.027 | ZrCl₄ (1.1) | iPrOH | 0 | 20 | 75 | 5 |
| C | Pt/C (5) | 0.003 | ZrCl₄ (1.1) | iPrOH | 0 | 50 | 50 | 0 |
| D | Pt/C (25) | 0.014 | TiCl₄ (1.1) | iPrOH | 0 | 25 | 75 | 0 |
| E | Pt/C (50) | 0.027 | TiCl₄ (1.1) | iPrOH | 0 | 15 | 80 | 5 |

Method 2: Similar to method 1, except that a 50 ml 316 SS steel autoclave without glass vial, 20 ml solvent (i-PrOH) and 600 mg (2.81 mmol) compound A1 are used. The reaction mixture is stirred overnight under the conditions (pressure/temperature) indicated below. The catalyst (5% w/w Pt/C) is from Johnson Matthey. The amount of catalyst and additive is relative to the compound A1.

| | | Catalyst | | | | Comp. | Comp. | Comp. | |
|---|---|---|---|---|---|---|---|---|---|
| | Catalyst | (mol. | Additive | Pressure | Temp. | A1 | A12 | A11 | Unknown |
| Experiment | (% w/w) | eq.) | (mol. eq.) | (bar) | (° C.) | (area %) | (area %) | (area %) | (area %) |
| F | Pt/C (5) | 0.003 | TiCl₄ (1.1) | 80 | 60 | 15 | 10 | 55 | 20 |
| G | Pt/C (5) | 0.003 | TiCl₄ (1.1) | 55 | 55 | 0 | 15 | 80 | 5 |

### Example 13

### Carbonyl to Hydroxy Reduction of Compound A1 Using H₂/cat. Ru/C

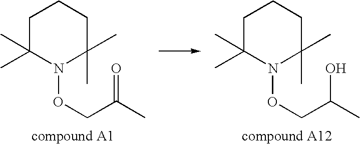

A mixture of 8 g (37.5 mmol) of the compound A1, 0.8 g Ru/C (5% Ru on carbon, 0.011 mol. eq.) and 50 ml methanol is hydrogenated at 50° C./45 bar during 2 hours. After filtration solvents are distilled off and the residue (7.4 g, 92%) distilled in a Kugelrohr to give 5.7 g (71%) of a liquid.

Anal. Calculated for C₁₂H₂₅NO₂ (215.34): C, 66.93; H, 11.70; N, 6.50. Found: C, 65.84; H, 10.98; N, 6.36.

¹H-NMR (400 MHz, CDCl₃), δ (ppm): 1.1-1.2 (m, 15H), 1.25-1.7 (m, 6H), 2.4 (br s, 1H; exchangeable proton), 3.66-3.75 (m, 2H), 3.96-4.04 (m, 1H).

### Example 14

### Carbonyl to Hydroxy Reduction of Compound A10 Using NaBH₄

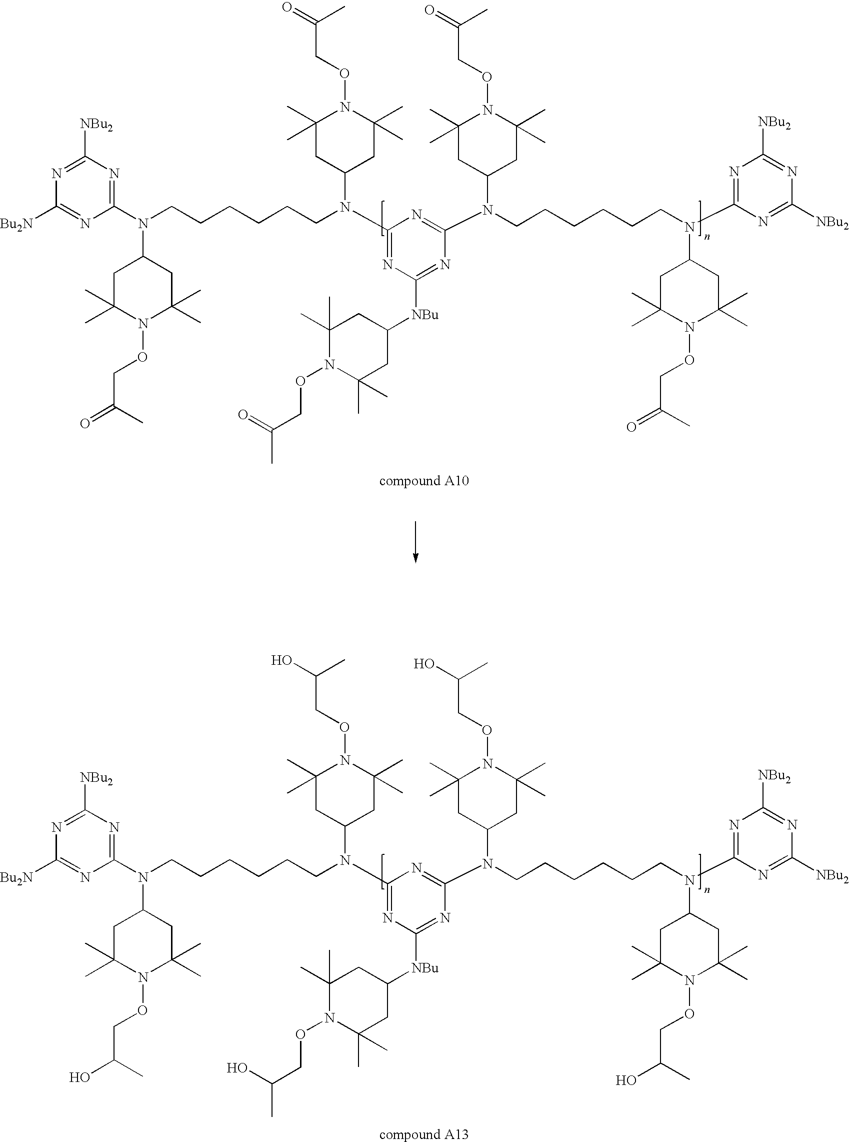

To a suspension of 0.93 g (23.5 mmol) NaBH₄ in 70 ml THF are added slowly at 25° C. a solution of 7 g (ca 2.8 meq NO—CH₂COCH₃/g) compound A10 in 70 ml THF. The mixture is brought to 45° C. and stirred for 5 minutes. A sample is withdrawn and analyzed by ¹H-NMR, indicating 100% conversion. The mixture is cooled down to 0° C. followed by the addition of 35 ml water. Ethylacetate is added, the aqueous phase split off and the organic phase water and brine washed. Evaporation of the solvent yields 6 g of a solid.

Anal. Found: C, 64.52; H, 10.15; N, 14.62; 0, 9.40.

¹H-NMR (300 MHz, CDCl₃), δ (ppm, NO—CH₂CH(OH)CH₃ only): 2.1-2.4 (br s, 1H, exchangeable proton) 3.65-3.8 (br m, 2H), 3.95-4.1 (br m, 1H).

### Application Examples

### Example 15

### Performance of Compound A13 as Flame Retardant in Polyolefin

Sample preparation: commercial polypropylene (Moplen HF 500 N®, manufacturer: Basell) is extruded on a twin-screw extruder ZSK 25 from Werner & Pfleiderer at a temperature of Tₘₐₓ=230° C. (heating zones 1-6), a throughput of 4 kg/h and 100 rpm with addition of basic-level stabilization (0.15% IRGANOX® B 225 and 0.05% calcium stearate. IRGANOX® B 225 is a 1:1 mixture of Irgafos®168 and IRGANOX® 1010) and stabilization by a compound of the invention (comparative example without). The resulting polymer is strand granulated in a water bath. The polymer is compressions moulded into 200 μm films at 230° C. The obtained films are tested according to DIN 4201-B2.

| | Weight | Length | |
|---|---|---|---|
| Additive | loss (%) | burnt (mm) | B2-classification |
| —* | 100 | 190 | failed |
| 0.5% compound A13 | 4.2 | 55 | passed |

Polymer density at 230° C = 0.75 g/cm³
*comparative example

### Example 16

### Performance of Compound A13 as Light Stabilizer in a Clear Coat

A compound of the present invention (1% based on the solids content of the formulation) and Tinvuin® 384 (2% based on the solids content of the formulation) is incorporated into a thermosetting acryl/melamine clear coat (based on Viacryl® SC 303/Viacryl® SC 370/Maprenal® MF 650) (solids content: 50.4%). The comparative example is without the compound of the present invention. The clear coat is sprayed onto silver metallic base coat resulting after cure (130° C./30′) in a dry film thickness of the clear coat of 40 μm. As a substrate electro coated aluminium panels (10×30 cm) as commercially available from ACT Laboratories (ACT Laboratories, Inc., Southfield, Mich. 48 075, USA) are being used. The panels are subsequently exposed in a QUV exposure device (Q-Panel Company using UV B 313 lamps. The 20° gloss is recorded in regular intervals. The test results are summarized in Table 1.

TABLE 1

| | 20°Gloss after . . . h of exposure | | | |
|---|---|---|---|---|
| Sample | 0 h | 1000 h | 1500 h | 2000 h |
| 1% compound A 13 | 89 | 89 | 87 | 85 |
| —* | 89 | 82 | 33 (end with cracking) | — |

*comparative example

### Clearcoat Formulation:

| | | | |
|---|---|---|---|
| a) | Viacryl SC 303¹ | 27.51 | g |
| | (65% solution in xylene/butanol, 26:9 wt./wt.) | | |
| b) | Viacryl SC 370² | 23.34 | g |
| | (75% in Solvesso 100³ | | |
| c) | Maprenal MF 650⁴ | 27.29 | g |
| | (55% in isobutanol) | | |
| d) | Butylacaetate/butanol (37:8 wt./wt.) | 4.33 | g |
| e) | Isobutanol | 4.87 | g |
| f) | Solvesso 150⁵ | 2.72 | g |
| g) | Crystal oil 30⁶ | 8.74 | g |
| h) | Baysilone MA⁷ (1% in Solvesso 150) | 1.20 | g |
| | Total | 100.00 | g |

Raw materials:
¹Viacryl SC 303: acrylic resin (Solutia, formerly Vianova Resins)
²Viacryl SC 370: acrylic resin (Solutia, formerly Vianova Resins)
³Solvesso 100: aromatic hydrocarbon, bp. 163-180° C. (Exxon Corp.)
⁴Maprenal MF 650: melamine resin (Solutia, formerly Vianova Resins)
⁵Solvesso 150: aromatic hydrocarbon, bp. 180-203° C. (Exxon Corp.)
⁶Crystal oil 30: aliphatic hydrocarbon, bp. 145-200° C. (Shell Corp.)
⁷Baysilone MA: leveling agent (Bayer AG)

1. A process for the preparation of a sterically hindered amine ether which comprises reacting a corresponding sterically hindered amine oxide with a ketone or an aldehyde with at least one reactive H in the presence of a peroxydisulphate.

2. A process according to claim 1, wherein the ketone or aldehyde contains at least two reactive H.

3. A process according to claim 1, wherein the reactive H is in α position.

4. A process according to claim 1, wherein the obtained product is subsequently hydrogenated.

5. A process according to claim 1, wherein the sterically hindered amine oxide contains at least one group of formula (100) 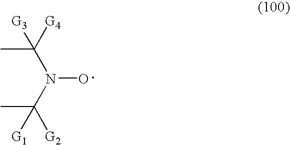
wherein G₁, G₂, G₃ and G₄ are independently alkyl of 1 to 4 carbon atoms or G₁ and G₂ and/or G₃ and G₄ are together tetramethylene or pentamethylene.

6. A process according to claim 1, wherein the obtained sterically hindered amine ether contains at least one group of formula (101) 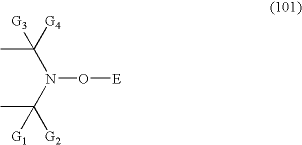
wherein G₁, G₂, G₃ and G₄ are independently alkyl of 1 to 4 carbon atoms or G₁ and G₂ and/or G₃ and G₄ are together tetramethylene or pentamethylene; 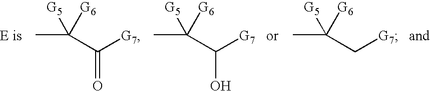
G₅, G₆ and G₇ are independently H, alkyl of 1 to 18 carbon atoms, alkenyl of 2 to 18 carbon atoms, alkinyl of 2 to 18 carbon atoms, aryl of 6 to 10 carbon atoms, aralkyl of 7 to 15 carbon atoms or COO(alkyl) of 2 to 19 carbon atoms; or
G₆ and G₇ form together alkylene of 3 to 10 carbon atoms.

7. A process according to claim 6, wherein the sterically hindered amine ether is of formula (A) to (O) 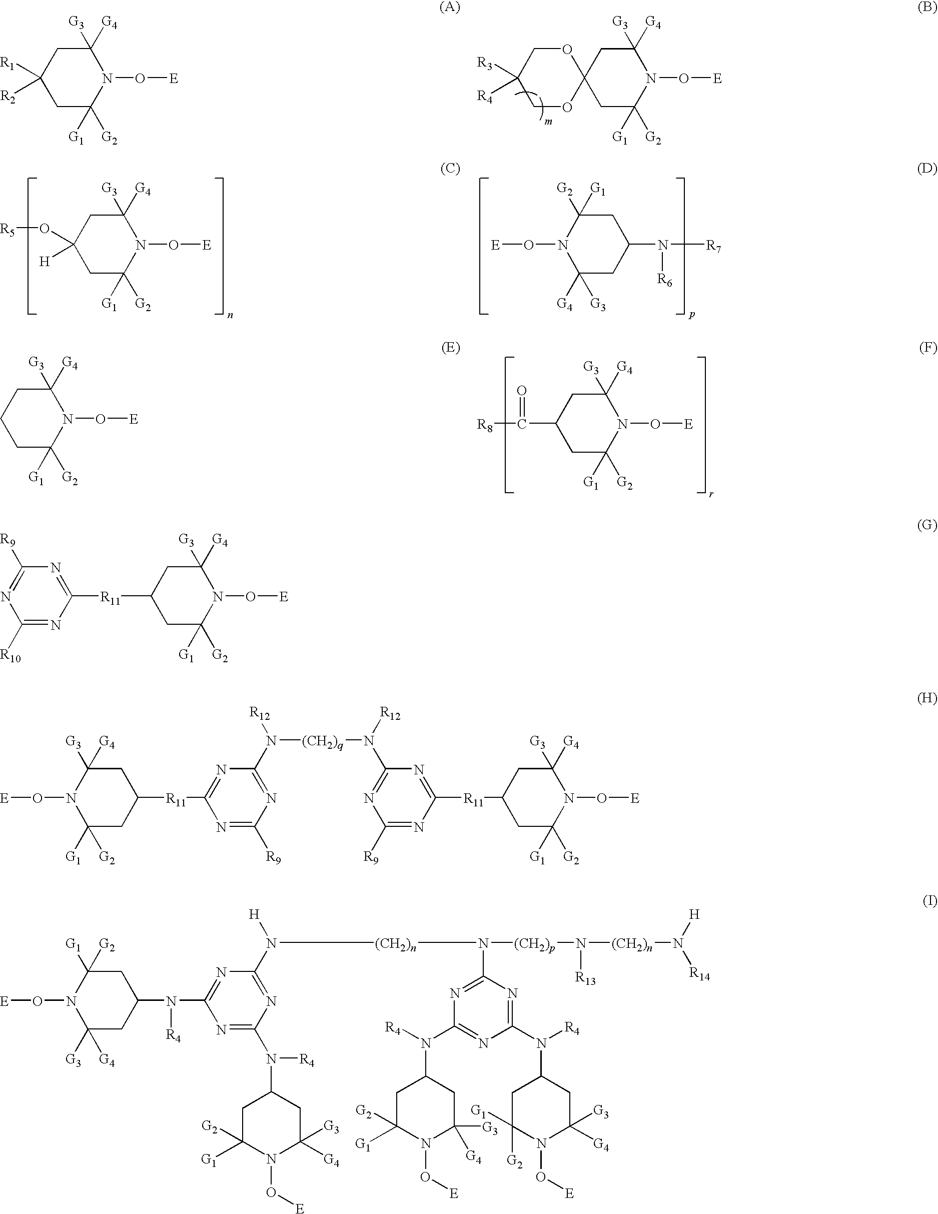 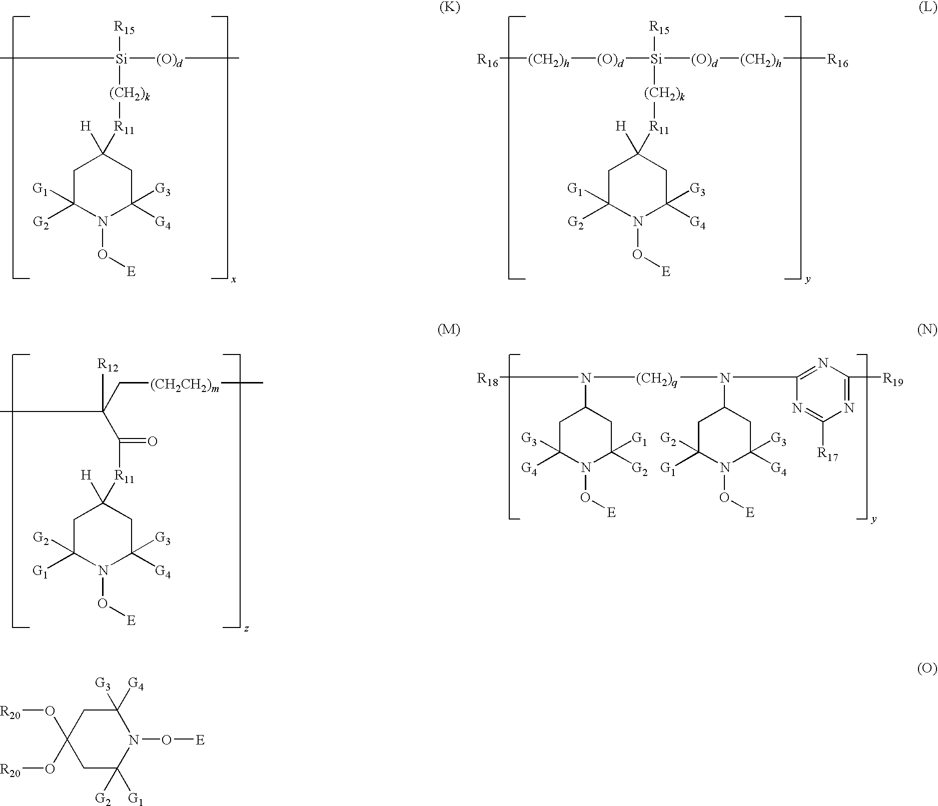
wherein G₁, G₂, G₃ and G₄ are independently alkyl of 1 to 4 carbon atoms or G₁ and G₂ and/or G₃ and G₄ are together tetramethylene or pentamethylene;
R₁ and R₂ are together ═O when E is 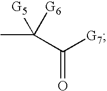
R₁ is H and R₂ is OH when E is 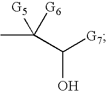
R₁ and R₂ are H when E is 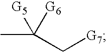
m is 0 or 1;
R₃ is hydrogen, hydroxyl or hydroxymethyl;
R₄ is hydrogen, alkyl of 1 to 12 carbon atoms or alkenyl of 2 to 12 carbon atoms;
n is 1 to 4;
when n is 1,
R₅ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms;
when n is 2,
R₅ is alkylene of 2 to 18 carbon atoms, a divalent acyl radical of an aliphatic or unsaturated aliphatic dicarboxylic or dicarbamic acid containing 2 to 18 carbon atoms, a divalent acyl radical of a cyclo-aliphatic dicarboxylic or dicarbamic acid containing 7 to 12 carbon atoms, or a divalent acyl radical of an aromatic dicarboxylic acid containing 8 to 15 carbon atoms;
when n is 3,
R₅ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
when n is 4,
R₅ is a tetravalent acyl radical of an aliphatic or unsaturated aliphatic tetracarboxylic acid, or R₅ is a tetravalent acyl radical of an aromatic tetracarboxylic acid containing 10 to 18 carbon atoms;
p is 1 to 3;
R₆ is hydrogen, alkyl of 1 to 18 carbon atoms or acyl of 2 to 6 carbon atoms or phenyl;
when p is 1,
R₇ is hydrogen, phenyl, alkyl of 1 to 18 carbon atoms, an acyl radical of an aliphatic or unsaturated aliphatic carboxylic or carbamic acid containing 2 to 18 carbon atoms, an acyl radical of a cyclo-aliphatic carboxylic or carbamic acid containing 7 to 12 carbon atoms, an acyl radical of an aromatic carboxylic acid containing 7 to 15 carbon atoms, or R₆ and R₇ together are —(CH₂)₅CO—, phthaloyl or a divalent acyl radical of maleic acid;
when p is 2,
R₇ is alkylene of 2 to 12 carbon atoms, a divalent acyl radical of an aliphatic or unsaturated aliphatic dicarboxylic or dicarbamic acid containing 2 to 18 carbon atoms, a divalent acyl radical of a cyclo-aliphatic dicarboxylic or dicarbamic acid containing 7 to 12 carbon atoms, or a divalent acyl radical of an aromatic dicarboxylic acid containing 8 to 15 carbon atoms;
when p is 3,
R₇ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
r is 1 to 4;
when r is 1,
R₈ is alkoxy of 1 to 18 carbon atoms, alkenyloxy of 2 to 18 carbon atoms, —NHalkyl of 1 to 18 carbon atoms or —N(alkyl)₂ of 2 to 36 carbon atoms;
when r is 2,
R₈ is alkylenedioxy of 2 to 18 carbon atoms, alkenylenedioxy of 2 to 18 carbon atoms, —NH-alkylene-NH— of 2 to 18 carbon atoms or —N(alkyl)-alkylene-N(alkyl)- of 2 to 18 carbon atoms, or R₈ is 4-methyl-1,3-phenylenediamino;
when r is 3,
R₈ is a trivalent alkoxy radical of a saturated or unsaturated aliphatic triol containing 3 to 18 carbon atoms;
when r is 4,
R₈ is a tetravalent alkoxy radical of a saturated or unsaturated aliphatic tetraol containing 4 to 18 carbon atoms;
R₉ and R₁₀ are independently chlorine, alkoxy of 1 to 18 carbon atoms, —O-T₁, amino substituted by 2-hydroxyethyl, —NH(alkyl) of 1 to 18 carbon atoms, —N(alkyl)T₁ with alkyl of 1 to 18 carbon atoms, or —N(alkyl)₂ of 2 to 36 carbon atoms;
R₁₁ is oxygen, or R₁₁ is nitrogen substituted by either hydrogen, alkyl of 1 to 12 carbon atoms or T₁;
T₁ is 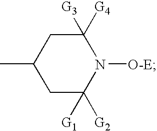
R₁₂ is hydrogen or methyl;
q is 2 to 8;
R₁₃ and R₁₄ are independently hydrogen or the group T₂; 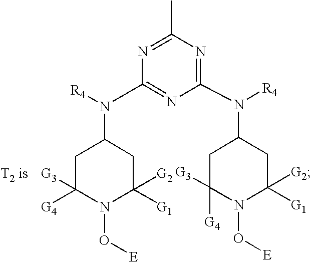
R₁₅ is hydrogen, phenyl, straight or branched alkyl of 1 to 12 carbon atoms, alkoxy of 1 to 12 carbon atoms, straight or branched alkyl of 1 to 4 carbon atoms substituted by phenyl, cycloalkyl of 5 to 8 carbon atoms, cycloalkenyl of 5 to 8 carbon atoms, alkenyl of 2 to 12 carbon atoms, glycidyl, allyloxy, straight or branched hydroxyalkyl of 1 to 4 carbon atoms, or silyl or silyloxy substituted three times independently by hydrogen, by phenyl, by alkyl of 1 to 4 carbon atoms or by alkoxy of 1 to 4 carbon atoms;
R₁₆ is hydrogen or silyl substituted three times independently by hydrogen, by phenyl, by alkyl of 1 to 4 carbon atoms or by alkoxy of 1 to 4 carbon atoms;
d is 0 or 1;
h is 0 to 4;
k is 0 to 5;
x is 3 to 6;
y is 1 to 10;
z is an integer such that the compound has a molecular weight of 1000 to 4000 amu, e.g. z may be from the range 3-10;
R₁₇ is morpholino, piperidino, 1-piperizinyl, alkylamino of 1 to 8 carbon atoms, —N(alkyl)T₁ with alkyl of 1 to 8 carbon atoms, or —N(alkyl)₂ of 2 to 16 carbon atoms;
R₁₈ is hydrogen, acyl of 2 to 4 carbon atoms, carbamoyl substituted by alkyl of 1 to 4 carbon atoms, s-triazinyl substituted once by chlorine and once by R₁₇, or s-triazinyl substituted twice by R₁₇ with the condition that the two R₁₇ substituents may be different;
R₁₉ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃;
T₃ is 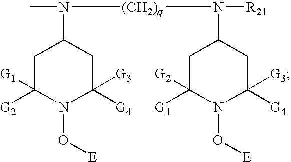
R₂₀ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms; and
R₂₁ is hydrogen, acyl of 2 to 4 carbon atoms, carbamoyl substituted by alkyl of 1 to 4 carbon atoms, s-triazinyl substituted twice by —N(alkyl)₂ of 2 to 16 carbon atoms or s-triazinyl substituted twice by —N(alkyl)T₁ with alkyl of 1 to 8 carbon atoms.

8. A process according to claim 1, wherein the process is carried out in the presence of a catalyst A.

9. A process according to claim 8, wherein the catalyst A is a salt or a complex of Ag, Mn, Fe, Cu, Co or Ni.

10. A process according to claim 1, wherein the process is carried out in the presence of a base.

11. A process according to claim 10, wherein the base is selected from the group consisting of alkali metal hydroxide, alkaline-earth metal hydroxide, alkali metal hydrogen carbonate, alkaline-earth metal hydrogen carbonate, alkali metal carbonate and alkaline-earth metal carbonate.

12. A process according to claim 4, wherein the hydrogenation is carried out in the presence of a catalyst B and H₂ or in the presence of a hydrogen equivalent.

13. A process according to claim 12, wherein the catalyst B is selected from the group consisting of Pd, Pt, PtO₂, Ru, Rh, Ir, Ni, mixtures thereof, salts thereof and complexes thereof and the hydrogenation is carried out in the presence or absence of an additive.

14. A process according to claim 12, wherein the hydrogen equivalent is a borohydride; a borane; an aluminumhydride; a silane; a secondary alkanol in combination with its alkoxide; a metal in combination with a protic solvent; an alkali metal hydride; formic acid, an alkali metal formiate or an ammonium formiate, each in combination with HCOOHₓNEt₃ or Pd/C; phosphinic acid, an alkali metal phosphinate or an ammonium phosphinate, each in combination with NaH₂PO₂ or Pd/C; an alkali metal dithionite; baker's yeast; or a borohydride, a borane, an aluminumhydride, a silane or a metal, each in combination with an additive.

15. A process according to claim 13 wherein the additive is a Lewis acid.

16. A sterically hindered amine ether of formula (A′) to (O′) 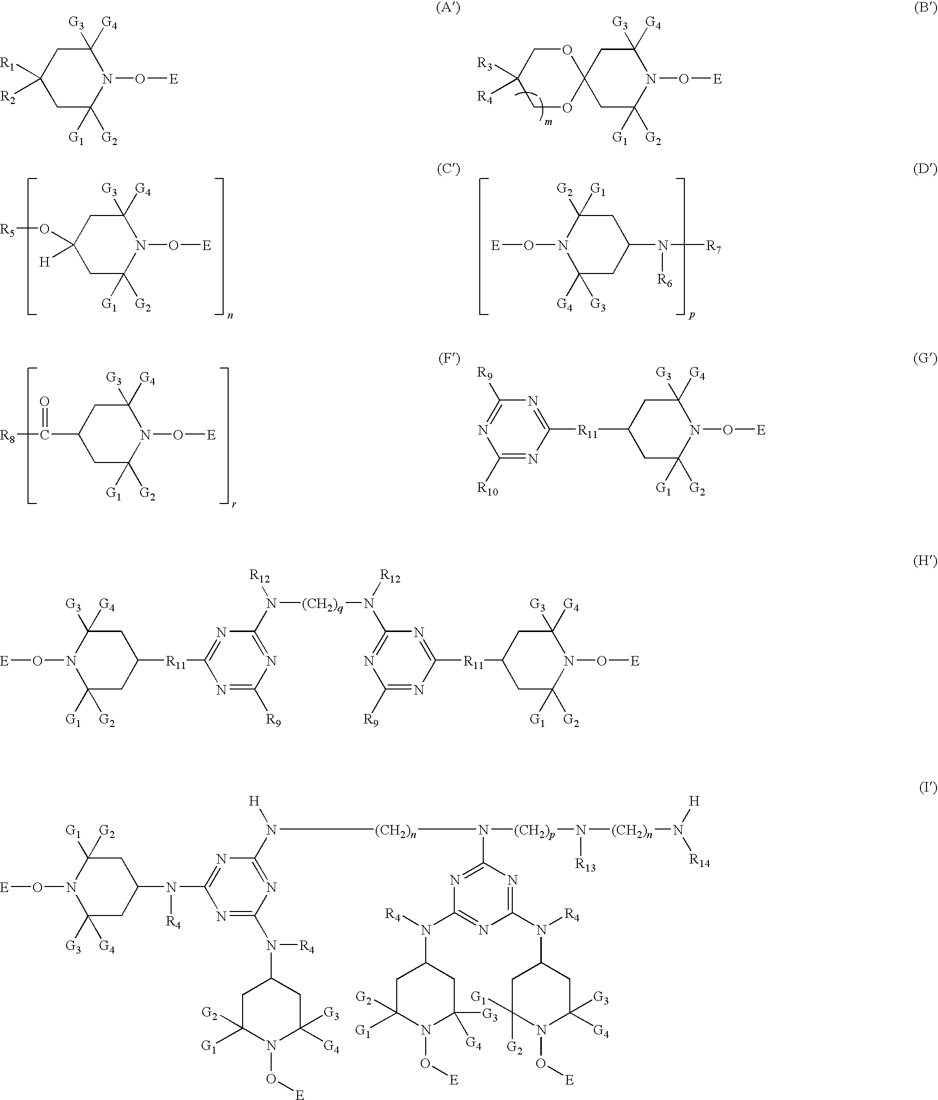 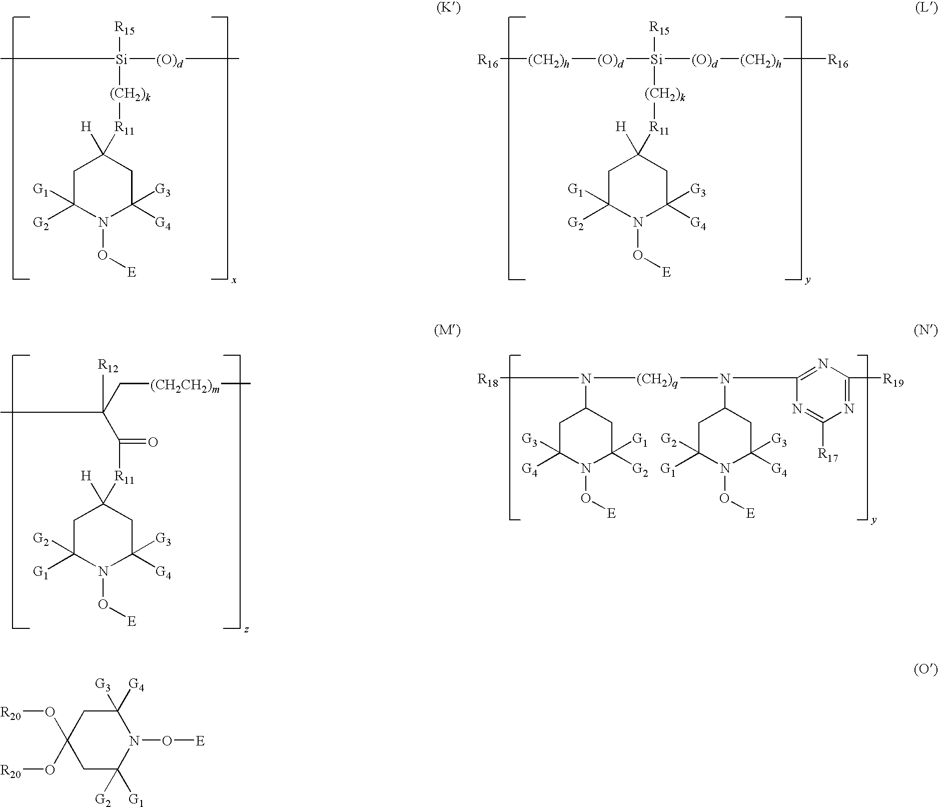
wherein G₁, G₂, G₃ and G₄ are independently alkyl of 1 to 4 carbon atoms or G₁ and G₂ and/or G₃ and G₄ are together tetramethylene or pentamethylene;
E is 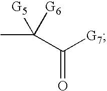
G₅, G₆ and G₇ are independently H, alkyl of 1 to 18 carbon atoms, alkenyl of 2 to 18 carbon atoms, alkinyl of 2 to 18 carbon atoms, aryl of 6 to 10 carbon atoms, aralkyl of 7 to 15 carbon atoms or COO(alkyl) of 2 to 19 carbon atoms; or
G₆ and G₇ form together alkylene of 3 to 10 carbon atoms;
R₁ and R₂ are together ═O;
m is 0 or 1;
R₃ is hydrogen, hydroxyl or hydroxymethyl;
R₄ is hydrogen, alkyl of 1 to 12 carbon atoms or alkenyl of 2 to 12 carbon atoms;
n is 1 to 4;
when n is 1,
R₅ is hydrogen, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cyclo-aliphatic carboxylic or carbamic acid containing 7 to 12 carbon atoms, or acyl radical of an aromatic acid containing 7 to 15 carbon atoms;
when n is 2,
R₅ is alkylene of 2 to 18 carbon atoms, a divalent acyl radical of an aliphatic or unsaturated aliphatic dicarboxylic or dicarbamic acid containing 2 to 18 carbon atoms, a divalent acyl radical of a cyclo-aliphatic dicarboxylic or dicarbamic acid containing 7 to 12 carbon atoms, or a divalent acyl radical of an aromatic dicarboxylic acid containing 8 to 15 carbon atoms;
when n is 3,
R₅ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
when n is 4,
R₅ is a tetravalent acyl radical of an aliphatic or unsaturated aliphatic tetracarboxylic acid, or R₅ is a tetravalent acyl radical of an aromatic tetracarboxylic acid containing 10 to 18 carbon atoms;
p is 1 to 3;
R₆ is hydrogen, alkyl of 1 to 18 carbon atoms or acyl of 2 to 6 carbon atoms or phenyl;
when p is 1,
R₇ is hydrogen, phenyl, alkyl of 1 to 18 carbon atoms, an acyl radical of an aliphatic or unsaturated aliphatic carboxylic or carbamic acid containing 2 to 18 carbon atoms, an acyl radical of a cyclo-aliphatic carboxylic or carbamic acid containing 7 to 12 carbon atoms, an acyl radical of an aromatic carboxylic acid containing 7 to 15 carbon atoms, or R₆ and R₇ together are —(CH₂)₅CO—, phthaloyl or a divalent acyl radical of maleic acid;
when p is 2,
R₇ is alkylene of 2 to 12 carbon atoms, a divalent acyl radical of an aliphatic or unsaturated aliphatic dicarboxylic or dicarbamic acid containing 2 to 18 carbon atoms, a divalent acyl radical of a cyclo-aliphatic dicarboxylic or dicarbamic acid containing 7 to 12 carbon atoms, or a divalent acyl radical of an aromatic dicarboxylic acid containing 8 to 15 carbon atoms;
when p is 3,
R₇ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
r is 1 to 4;
when r is 1,
R₈ is alkoxy of 1 to 18 carbon atoms, alkenyloxy of 2 to 18 carbon atoms, —NHalkyl of 1 to 18 carbon atoms or —N(alkyl)₂ of 2 to 36 carbon atoms;
when r is 2,
R₈ is alkylenedioxy of 2 to 18 carbon atoms, alkenylenedioxy of 2 to 18 carbon atoms, —NH-alkylene-NH— of 2 to 18 carbon atoms or —N(alkyl)-alkylene-N(alkyl)- of 2 to 18 carbon atoms, or R₈ is 4-methyl-1,3-phenylenediamino;
when r is 3,
R₈ is a trivalent alkoxy radical of a saturated or unsaturated aliphatic triol containing 3 to 18 carbon atoms;
when r is 4,
R₈ is a tetravalent alkoxy radical of a saturated or unsaturated aliphatic tetraol containing 4 to 18 carbon atoms;
R₉ and R₁₀ are independently chlorine, alkoxy of 1 to 18 carbon atoms, —O-T₁, amino substituted by 2-hydroxyethyl, —NH(alkyl) of 1 to 18 carbon atoms, —N(alkyl)T₁ with alkyl of 1 to 18 carbon atoms, or —N(alkyl)₂ of 2 to 36 carbon atoms;
R₁₁ is oxygen, or R₁₁ is nitrogen substituted by either hydrogen, alkyl of 1 to 12 carbon atoms or T₁;
T₁ is 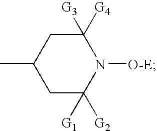
R₁₂ is hydrogen or methyl;
q is 2 to 8;
R₁₃ and R₁₄ are independently hydrogen or the group T₂;
T₂ is 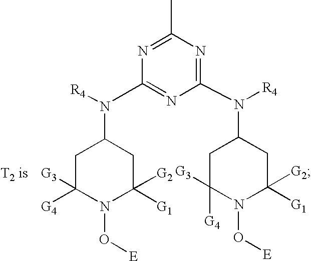
R₁₅ is hydrogen, phenyl, straight or branched alkyl of 1 to 12 carbon atoms, alkoxy of 1 to 12 carbon atoms, straight or branched alkyl of 1 to 4 carbon atoms substituted by phenyl, cycloalkyl of 5 to 8 carbon atoms, cycloalkenyl of 5 to 8 carbon atoms, alkenyl of 2 to 12 carbon atoms, glycidyl, allyloxy, straight or branched hydroxyalkyl of 1 to 4 carbon atoms, or silyl or silyloxy substituted three times independently by hydrogen, by phenyl, by alkyl of 1 to 4 carbon atoms or by alkoxy of 1 to 4 carbon atoms;
R₁₆ is hydrogen or silyl substituted three times independently by hydrogen, by phenyl, by alkyl of 1 to 4 carbon atoms or by alkoxy of 1 to 4 carbon atoms;
d is 0 or 1;
h is 0 to 4;
k is 0 to 5;
x is 3 to 6;
y is 1 to 10;
z is an integer such that the compound has a molecular weight of 1000 to 4000 amu, e.g. z may be from the range 3-10;
R₁₇ is morpholino, piperidino, 1-piperizinyl, alkylamino of 1 to 8 carbon atoms, —N(alkyl)T₁ with alkyl of 1 to 8 carbon atoms, or —N(alkyl)₂ of 2 to 16 carbon atoms;
R₁₈ is hydrogen, acyl of 2 to 4 carbon atoms, carbamoyl substituted by alkyl of 1 to 4 carbon atoms, s-triazinyl substituted once by chlorine and once by R₁₇, or s-triazinyl substituted twice by R₁₇ with the condition that the two R₁₇ substituents may be different;
R₁₉ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃;
T₃ is 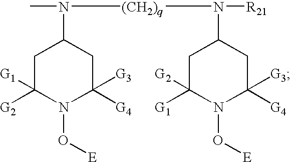
R₂₀ is hydrogen, alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms; and
R₂₁ is hydrogen, acyl of 2 to 4 carbon atoms, carbamoyl substituted by alkyl of 1 to 4 carbon atoms, s-triazinyl substituted twice by —N(alkyl)₂ of 2 to 16 carbon atoms or s-triazinyl substituted twice by —N(alkyl)T₁ with alkyl of 1 to 8 carbon atoms
with the proviso
that in formula (A′), G₇ is not methyl when G₅ and G₆ are H and
that in formula (C′), R₅ is not benzoyl when n is 1.

17. A sterically hindered amine ether according to claim 16 of formula (A′), (C′) or (N′), wherein
n is 1 to 2;
when n is 1,
R₅ is hydrogen or an acyl radical of an aliphatic or unsaturated aliphatic carboxylic or carbamic acid containing 2 to 18 carbon atoms;
when n is 2,
R₅ is alkylene of 2 to 18 carbon atoms, a divalent acyl radical of an aliphatic or unsaturated aliphatic dicarboxylic or dicarbamic acid containing 2 to 18 carbon atoms;
q is 4 to 8;
y is 2 to 10;
R₁₇ is alkylamino of 1 to 8 carbon atoms, —N(alkyl)T₁ with alkyl of 1 to 8 carbon atoms, or —N(alkyl)₂ of 2 to 16 carbon atoms;
R₁₈ is hydrogen, s-triazinyl substituted once by chlorine and once by R₁₇, or s-triazinyl substituted twice by R₁₇ with the condition that the two R₁₇ substituents may be different;
R₁₉ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃;
T₃ is 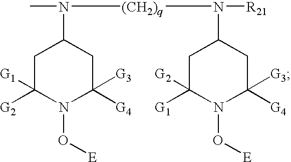
and
R₂₁ is hydrogen, s-triazinyl substituted twice by —N(alkyl)₂ of 2 to 16 carbon atoms or s-triazinyl substituted twice by —N(alkyl)T₁ with alkyl of 1 to 8 carbon atoms.

18. A sterically hindered amine ether of formula (Z′) 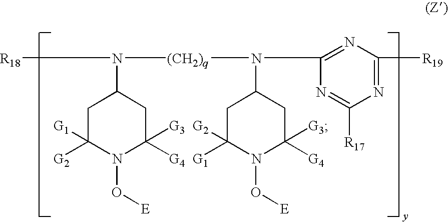
wherein E is 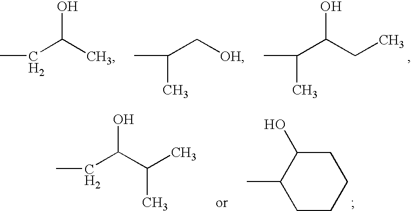
G₁, G₂, G₃ and G₄ are independently alkyl of 1 to 4 carbon atoms or G₁ and G₂ and/or G₃ and G₄ are together tetramethylene or pentamethylene;
q is 2 to 8;
R₁₇ is morpholino, piperidino, 1-piperizinyl, alkylamino of 1 to 8 carbon atoms, —N(alkyl)T₁ with alkyl of 1 to 8 carbon atoms, or —N(alkyl)₂ of 2 to 16 carbon atoms;
R₁₈ is hydrogen, acyl of 2 to 4 carbon atoms, carbamoyl substituted by alkyl of 1 to 4 carbon atoms, s-triazinyl substituted once by chlorine and once by R₁₇, or s-triazinyl substituted twice by R₁₇ with the condition that the two R₁₇ substituents may be different;
R₁₉ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃; T₃ is 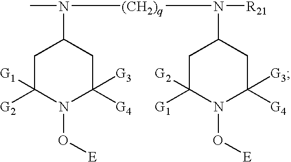
R₂₁ is hydrogen, acyl of 2 to 4 carbon atoms, carbamoyl substituted by alkyl of 1 to 4 carbon atoms, s-triazinyl substituted twice by —N(alkyl)₂ of 2 to 16 carbon atoms or s-triazinyl substituted twice by —N(alkyl)T₁ with alkyl of 1 to 8 carbon atoms;
and
T₁ is 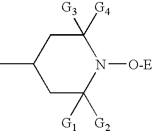
.

19. A composition comprising
A) an organic polymer which is sensitive to oxidative, thermal and/or actinic degradation, and
B) at least one sterically hindered amine ether as defined in claim 16.

20. (canceled)

21. A composition according to claim 19, comprising further additives selected from phenolic antioxidants, aminic antioxidants, hindered amine light stabilizers, UV-absorbers, phosphites, phosphonites, benzofuranones, metal stearates, metal oxides, pigments, dyes, organophophorus compounds, hydroxylamines, flame retardants and mixtures thereof.

22. A process for flame retarding an organic polymer or stabilizing an organic polymer against degradation by light, oxygen and/or heat, which process comprises applying to or incorporating into said polymer at least one sterically hindered amine ether as defined in claim 16.

23. (canceled)

24. A composition comprising
A) an organic polymer which is sensitive to oxidative, thermal and/or actinic degradation, and
B) at least one sterically hindered amine ether as defined in claim 18.

25. A composition according to claim 24, comprising further additives selected from phenolic antioxidants, aminic antioxidants, hindered amine light stabilizers, UV-absorbers, phosphites, phosphonites, benzofuranones, metal stearates, metal oxides, pigments, dyes, organophophorus compounds, hydroxylamines, flame retardants and mixtures thereof.

26. A process for flame retarding an organic polymer or stabilizing an organic polymer against degradation by light, oxygen and/or heat, which process comprises applying to or incorporating into said polymer at least one sterically hindered amine ether as defined in claim 18.
